SEQUENCE LISTING (27 sequences)

SEQ ID NO: 1 - protein, 304 aa, synthetic construct
  REGION 1..304: note = UniProtKB accession P02686-1
MGNHAGKRELNAEKASTNSETNRGESEKKRNLGELSRTTSEDNEVFGEADANQNNGTSSQDTAVTDSKRTADPKNAWQDAHPADPGSRPHLIRLFSRDAPGREDNTFKDRPSESDELQTIQEDSAATSESLDVMASQKRPSQRHGSKYLATASTMDHARHGFLPRHRDTGILDSIGRFFGGDRGAPKRGSGKDSHHPARTAHYGSLPQKSHGRTQDENPVVHFFKNIVTPRTPPPSQGKGRGLSLSRFSWGAEGQRPGFGYGGRASDYKSAHKGFKGVDAQGTLSKIFKLGGRDSRSGSPMARR

SEQ ID NO: 2 - protein, 41 aa, synthetic construct
  REGION 1..41: note = MBP 100-140
PPSQGKGRGLSLSRFSWGAEGQRPGFGYGGRASDYKSAHKG

SEQ ID NO: 3 - protein, 19 aa, synthetic construct
  REGION 1..19: note = MBP 111-129
LSRFSWGAEGQRPGFGYGG

SEQ ID NO: 4 - protein, 8 aa, synthetic construct
  REGION 1..8: note = MBP 116-123
WGAEGQRP

SEQ ID NO: 5 - protein, 28 aa, synthetic construct
  REGION 1..28: note = CDR3alpha
TVYGGATNKLIFGTGTLLAVQPNIQNPD

SEQ ID NO: 6 - protein, 23 aa, synthetic construct
  REGION 1..23: note = CDR3beta
SARGGSYNSPLHFGNGTRLTVTE

SEQ ID NO: 7 - protein, 7 aa, synthetic construct
  REGION 1..7: note = CDR1alpha
TISGTDY

SEQ ID NO: 8 - protein, 5 aa, synthetic construct
  REGION 1..5: note = CDR2alpha
GLTSN

SEQ ID NO: 9 - protein, 28 aa, synthetic construct
  REGION 1..28: note = CDR3alpha
TVYGGATNKLIFGTGTLLAVQPNIQNPD

SEQ ID NO: 10 - protein, 6 aa, synthetic construct
  REGION 1..6: note = CDR1beta
DFQATT

SEQ ID NO: 11 - protein, 7 aa, synthetic construct
  REGION 1..7: note = CDR2beta
SNEGSKA

SEQ ID NO: 12 - protein, 23 aa, synthetic construct
  REGION 1..23: note = CDR3beta
SARGGSYNSPLHFGNGTRLTVTE

SEQ ID NO: 13 - protein, 250 aa, synthetic construct
  REGION 1..250: note = Human alpha V-region and murine constant-region
DAKTTQPNSMESNEEEPVHLPCNHSTISGTDYIHWYRQLPSQGPEYVIHGLTSNVNNRMASLAIAEDRKSSTLILHRATLRDAAVYYCTVYGGATNKLIFGTGTLLAVQPNIQNPDPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLSVMGLRILLLKVAGFNLLMTLRLWSSGSG

SEQ ID NO: 14 - protein, 289 aa, synthetic construct
  REGION 1..289: note = Human beta V-region and murine constant-region
GAVVSQHPSWVIAKSGTSVKIECRSLDFQATTMFWYRQFPKQSLMLMATSNEGSKATYEQGVEKDKFLINHASLTLSTLTVTSAHPEDSSFYICSARGGSYNSPLHFGNGTRLTVTEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYHQGVLSATILYEILLGKATLYAVLVSGLVLMAMVKKKNS

SEQ ID NO: 15 - protein, 17 aa, synthetic construct
  REGION 1..17: note = Leader sequence
MKLVTSITVLLSLGIMG

SEQ ID NO: 16 - protein, 25 aa, synthetic construct
  REGION 1..25: note = Leader sequence
MLLLLLLLGPGISLLLPGSLAGSGL

SEQ ID NO: 17 - protein, 431 aa, synthetic construct
  REGION 1..431: note = FOXP3 polypeptide - UniProtKB accession: Q9BZS1
MPNPRPGKPSAPSLALGPSPGASPSWRAAPKASDLLGARGPGGTFQGRDLRGGAHASSSSLNPMPPSQLQLPTLPLVMVAPSGARLGPLPHLQALLQDRPHFMHQLSTVDAHARTPVLQVHPLESPAMISLTPPTTATGVFSLKARPGLPPGINVASLEWVSREPALLCTFPNPSAPRKDSTLSAVPQSSYPLLANGVCKWPGCEKVFEEPEDFLKHCQADHLLDEKGRAQCLLQREMVQSLEQQLVLEKEKLSAMQAHLAGKMALTKASSVASSDKGSCCIVAAGSQGPVVPAWSGPREAPDSLFAVRRHLWGSHGNSTFPEFLHNMDYFKFHNMRPPFTYATLIRWAILEAPEKQRTLNEIYHWFTRMFAFFRNHPATWKNAIRHNLSLHKCFVRVESEKGAVWTVDELEFRKKRSQRPSRCSNPTPGP

SEQ ID NO: 18 - DNA, 408 bp, synthetic construct
  misc_feature 1..408: note = Alpha Chain: V-region, MS2.3C8-alpha-variable(Va26-2) nucleotide sequence
  source 1..408: mol_type = other DNA
cccggaccgatgaagctcgtgaccagcatcaccgtgctgctgagcctgggcattatgggcgacgccaagaccacccagcccaacagcatggaaagcaacgaagaggaacccgtgcatctgccctgcaaccacagcaccatcagcggcaccgactacatccactggtacagacagctgcccagccagggccccgagtatgtgatccacggcctgaccagcaacgtgaacaaccggatggcctccctggccattgccgaggacagaaagagcagcaccctgatcctgcaccgggccaccctgagagatgccgccgtgtactactgcaccgtgtacggcggagccaccaacaagctgatcttcggcaccggcacactgctggccgtgcagcccaatatccagaaccccgac

SEQ ID NO: 19 - protein, 116 aa, synthetic construct
  REGION 1..116: note = Alpha Chain: V-region, MS2.3C8-alpha-variable(Va26-2) amino acid sequence
DAKTTQPNSMESNEEEPVHLPCNHSTISGTDYIHWYRQLPSQGPEYVIHGLTSNVNNRMASLAIAEDRKSSTLILHRATLRDAAVYYCTVYGGATNKLIFGTGTLLAVQPNIQNPD

SEQ ID NO: 20 - DNA, 423 bp, synthetic construct
  misc_feature 1..423: note = MS2.3C8 Beta Chain: human V-region nucleotide sequence
  source 1..423: mol_type = other DNA
atgcttctccttctgctgctgctgggacccggcatctcactgctgctgcctggatctctggccggctctggactgggagctgtggtgtctcagcacccctcttgggtcatcgccaagagcggcaccagcgtgaagatcgagtgcagaagcctggacttccaggccaccaccatgttttggtacaggcagttccccaagcagagcctgatgctgatggccacctccaacgagggcagcaaggccacatatgagcagggcgtggaaaaggacaagttcctgatcaaccacgccagcctgaccctgtccaccctgacagtgacaagcgcccaccccgaggactccagcttctacatctgtagcgccagaggcggcagctacaacagccctctgcactttggcaacggcacccggctgaccgtgacc

SEQ ID NO: 21 - protein, 111 aa, synthetic construct
  REGION 1..111: note = MS2.3C8 Beta Chain: human V-region amino acid sequence
GAVVSQHPSWVIAKSGTSVKIECRSLDFQATTMFWYRQFPKQSSCSWPPPTRAPRPPTSRASRRTSSSSTTPPSPSPPSPSPPPTPRTPPSTSAPPAAAPTTPPSTSATAP

SEQ ID NO: 22 - protein, 76 aa, synthetic construct
  REGION 1..76: note = MS2.3C8-alpha-variable amino acid sequence excluding CDR sequences
DAKTTQPNSMESNEEEPVHLPCNHSIHWYRQLPSQGPEYVIHVNNRMASLAIAEDRKSSTLILHRATLRDAAVYYC

SEQ ID NO: 23 - protein, 81 aa, synthetic construct
  REGION 1..81: note = MS2.3C8-beta-variable amino acid sequence excluding CDR sequences
GAVVSQHPSWVIAKSGTSVKIECRSLMFWYRQFPKQSLMLMATTYEQGVEKDKFLINHASLTLSTLTVTSAHPEDSSFYIC

SEQ ID NO: 24 - DNA, 1352 bp, synthetic construct
  misc_feature 1..1352: note = Nucleic acid sequence encoding FOXP3
  source 1..1352: mol_type = other DNA
gaattcgtcgacatgcccaaccccagacccggcaagccttctgccccttctctggccctgggaccatctcctggcgcctccccatcttggagagccgcccctaaagccagcgatctgctgggagctagaggccctggcggcacattccagggcagagatctgagaggcggagcccacgcctctagcagcagcctgaatcccatgccccctagccagctgcagctgcctacactgcctctcgtgatggtggcccctagcggagctagactgggccctctgcctcatctgcaggctctgctgcaggaccggccccactttatgcaccagctgagcaccgtggacgcccacgccagaacacctgtgctgcaggtgcaccccctggaaagccctgccatgatcagcctgacccctccaaccacagccaccggcgtgttcagcctgaaggccagacctggactgccccctggcatcaatgtggccagcctggaatgggtgtcccgcgaacctgccctgctgtgcaccttccccaatcctagcgcccccagaaaggacagcacactgtctgccgtgccccagagcagctatcccctgctggctaacggcgtgtgcaagtggcctggctgcgagaaggtgttcgaggaacccgaggacttcctgaagcactgccaggccgaccatctgctggacgagaaaggcagagcccagtgcctgctgcagcgcgagatggtgcagtccctggaacagcagctggtgctggaaaaagaaaagctgagcgccatgcaggcccacctggccggaaagatggccctgacaaaagccagcagcgtggccagctccgacaagggcagctgttgtatcgtggccgctggcagccagggacctgtggtgcctgcttggagcggacctagagaggcccccgatagcctgtttgccgtgcggagacacctgtggggcagccacggcaactctaccttccccgagttcctgcacaacatggactacttcaagttccacaacatgaggccccccttcacctacgccaccctgatcagatgggccattctggaagcccccgagaagcagcggaccctgaacgagatctaccactggtttacccggatgttcgccttcttccggaaccaccccgccacctggaagaacgccatccggcacaatctgagcctgcacaagtgcttcgtgcgggtggaaagcgagaagggcgccgtgtggacagtggacgagctggaatttcggaagaagcggtcccagaggcccagccggtgtagcaatcctacacctggccctgagggcagaggaagtctgctaacatgcggtgacgtcgaggagaatcc

SEQ ID NO: 25 - protein, 441 aa, synthetic construct
  REGION 1..441: note = Amino acid sequence of FOXP3
MPNPRPGKPSAPSLALGPSPGASPSWRAAPKASDLLGARGPGGTFQGRDLRGGAHASSSSLNPMPPSQLQLPTLPLVMVAPSGARLGPLPHLQALLQDRPHFMHQLSTVDAHARTPVLQVHPLESPAMISLTPPTTATGVFSLKARPGLPPGINVASLEWVSREPALLCTFPNPSAPRKDSTLSAVPQSSYPLLANGVCKWPGCEKVFEEPEDFLKHCQADHLLDEKGRAQCLLQREMVQSLEQVEELSAMQAHLAGKMALTKASSVASSDKGSCCIVAAGSQGPVVPAWSGPREAPDSLFAVRRHLWGSHGNSTFPEFLHNMDYFKFHNMRPPFTYATLIRWAILEAPEKQRTLNEIYHWFTRMFAFFRNHPATWKNAIRHNLSLHKCFVRVESEKGAVWTVDELEFRKKRSQRPSRCSNPTPGPEGRGSLLTCGDVEEN

SEQ ID NO: 26 - protein, 171 aa, synthetic construct
  REGION 1..171: note = UniProtKB accession P02686-5
MASQKRPSQRHGSKYLATASTMDHARHGFLPRHRDTGILDSIGRFFGGDRGAPKRGSGKDSHHPARTAHYGSLPQKSHGRTQDENPVVHFFKNIVTPRTPPPSQGKGRGLSLSRFSWGAEGQRPGFGYGGRASDYKSAHKGFKGVDAQGTLSKIFKLGGRDSRSGSPMARR

SEQ ID NO: 27 - DNA, 1296 bp, synthetic construct
  misc_feature 1..1296: note = FOXP3 polynucleotide sequence
  source 1..1296: mol_type = other DNA
atgcccaaccccaggcctggcaagccctcggccccttccttggcccttggcccatccccaggagcctcgcccagctggagggctgcacccaaagcctcagacctgctgggggcccggggcccagggggaaccttccagggccgagatcttcgaggcggggcccatgcctcctcttcttccttgaaccccatgccaccatcgcagctgcagctgcccacactgcccctagtcatggtggcaccctccggggcacggctgggccccttgccccacttacaggcactcctccaggacaggccacatttcatgcaccagctctcaacggtggatgcccacgcccggacccctgtgctgcaggtgcaccccctggagagcccagccatgatcagcctcacaccacccaccaccgccactggggtcttctccctcaaggcccggcctggcctcccacctgggatcaacgtggccagcctggaatgggtgtccagggagccggcactgctctgcaccttcccaaatcccagtgcacccaggaaggacagcaccctttcggctgtgccccagagctcctacccactgctggcaaatggtgtctgcaagtggcccggatgtgagaaggtcttcgaagagccagaggacttcctcaagcactgccaggcggaccatcttctggatgagaagggcagggcacaatgtctcctccagagagagatggtacagtctctggagcagcagctggtgctggagaaggagaagctgagtgccatgcaggcccacctggctgggaaaatggcactgaccaaggcttcatctgtggcatcatccgacaagggctcctgctgcatcgtagctgctggcagccaaggccctgtcgtcccagcctggtctggcccccgggaggcccctgacagcctgtttgctgtccggaggcacctgtggggtagccatggaaacagcacattcccagagttcctccacaacatggactacttcaagttccacaacatgcgaccccctttcacctacgccacgctcatccgctgggccatcctggaggctccagagaagcagcggacactcaatgagatctaccactggttcacacgcatgtttgccttcttcagaaaccatcctgccacctggaagaacgccatccgccacaacctgagtctgcacaagtgctttgtgcgggtggagagcgagaagggggctgtgtggaccgtggatgagctggagttccgcaagaaacggagccagaggcccagcaggtgttccaaccctacacctggcccctga

(19) United States
(12) Patent Application Publication
(10) Pub. No.: US 2025/0154532 A1
(43) Pub. Date: May 15, 2025

(54) ENGINEERED REGULATORY T CELL

(71) Applicant: UCL Business Ltd, London (GB)

(72) Inventors: Hans Stauss, London (GB); Graham P. Wright, London (GB); Jenny L. McGovern, London (GB)

(21) Appl. No.: 19/025,738

(22) Filed: Jan. 16, 2025

Related U.S. Application Data
(63) Continuation of application No. 17/048,587, filed on Oct. 17, 2020, now Pat. No. 12,227,756.

(30) Foreign Application Priority Data
Apr. 18, 2018 (GB) 1806330.5
Apr. 18, 2018 (GB) 1806331.3

(51) Int. Cl. C12N 15/87; A61K 35/12; A61K 35/17; A61K 38/00; A61K 40/11; A61K 40/22; A61K 40/32; A61K 40/41; C07K 14/725; C07K 14/74; C12N 5/0783

(52) U.S. Cl. CPC C12N 15/87; A61K 35/17; A61K 40/11; A61K 40/22; A61K 40/32; A61K 40/416; C07K 14/7051; C07K 14/70539; C12N 5/0637; A61K 2035/122; A61K 38/00; C12N 2510/00

(57) ABSTRACT

The present invention relates to an engineered regulatory T cell (Treg) comprising a T cell receptor (TCR) which is capable of specifically binding to a myelin basic protein (MBP) peptide or variant or fragment thereof when the peptide is presented by a major histocompatibility complex (MHC) molecule. The present invention further relates to methods for providing an engineered Treg and to methods and uses of said engineered Treg and vectors and kits of vectors encoding said Treg.

### CROSS-REFERENCE TO RELATED APPLICATIONS

This application is a continuation of U.S. patent application Ser. No. 17/048,587, filed on Oct. 17, 2020, which is a National Stage of International Application No. PCT/GB2019/051097, filed on Apr. 17, 2019, which claims the benefit of priority to GB Patent Application No. 1806330.5, filed on Apr. 18, 2018, to GB Patent Application No. 1806331.3, filed on Apr. 18, 2018, each of which is hereby incorporated by reference in its entirety for all purposes.

### SEQUENCE LISTING

The instant application contains a Sequence Listing which has been submitted electronically in XML format and is hereby incorporated by reference in its entirety. Said XML copy, created on Jan. 14, 2025, is named P112692USM and is 40,459 bytes in size.

### FIELD OF THE INVENTION

The present invention relates to an engineered regulatory T cell (Treg). In particular, the present invention relates to a Treg comprising a T cell receptor (TCR) which is capable of specifically binding to myelin basic protein (MBP). The present invention further relates to methods for providing an engineered Treg and to methods and uses of said engineered Treg.

### BACKGROUND TO THE INVENTION

Many autoimmune and inflammatory central nervous system (CNS) diseases are Involve autoreactive T-cells. For example, Multiple Sclerosis (MS), which is an autoimmune inflammatory demyelinating condition of the central nervous system and is the most common neurological disorder among young adults.

Current treatments for autoimmune and inflammatory CNS diseases generally suppress the immune system. For example, one treatment includes transplantation of bone marrow along with administration of cytostatics and immunosupressive drugs. Autologous haematopoietic stem cell transplantation can have lasting beneficial effects for some patients, but the procedure requires aggressive myelo-ablative conditioning which is associated with substantial toxicity and risk.

Although several disease-modifying treatments (DMTs) have been approved to reduce the frequency of clinical relapses, most patients continue to clinically deteriorate under current therapy schedules. Neither DMTs nor stem cell transplantation can mediate CNS-specific suppression of the immunopathology of autoimmune and inflammatory CNS diseases.

Currently, effective treatments for autoimmune and inflammatory CNS diseases do not exist. Treatment is focused on merely reducing its symptoms, usually by general suppression of the immune system. There is a need for a therapy which specifically targets local immune responses associated with onset and progression of CNS disease.

### SUMMARY OF ASPECTS OF THE INVENTION

The present invention is based, at least in part, on the inventors' determination that T cell receptor gene transfer technology can be used to generate antigen-specific Tregs. It has been shown that human antigen-specific Tregs can suppress activated T cells.

In particular, the present inventors have produced MBP-specific Tregs for example, by retroviral transfer of MBP-TCR genes into purified Tregs and by retroviral transfer of MBP-TCR and forkhead box P3 (FOXP3) genes into conventional CD4⁺ T cells. Without wishing to be bound by theory, these engineered Tregs with TCRs specific for MBP may be used in the suppression of diseases e.g. autoimmune diseases, where local activation of MBP-specific Tregs in the central nervous system (CNS) may suppress CNS pathology as seen in MS and other CNS inflammatory conditions.

Without wishing to be bound by theory, it was unexpected that the present inventors have been able to develop a Treg which suppresses proliferation of pathogenic T cells. It was previously suggested in the field that TCRs in Tregs have higher affinity for self antigen than TCRs in conventional T cells (Pacholczyk and Kern Immunology, 2008. 125(4) 450-458 incorporated herein by reference). It has also been reported that Tregs transduced with an islet-antigen specific TCR were less efficient than Tregs expressing a viral antigen specific TCR. It was suggested that this may be due Treg-specific TCR requirements—for example a certain affinity requirement. Thus, the Treg repertoire is highly diverse and was thought to have a distinct set of T cell receptors compared to the repertoire of conventional T cells. The present inventors have unexpectedly demonstrated that a MBP-specific TCR isolated from conventional T cells can be successfully expressed in a Treg cell and can produce a functional Treg.

Accordingly, the present invention provides an engineered regulatory T cell (Treg) comprising a T cell receptor (TCR) which is capable of specifically binding to a peptide which comprises at least 90% identity to MBP 111-129 (SEQ ID NO: 3) or a fragment thereof when the peptide is presented by a major histocompatibility complex (MHC) molecule.

The MBP111-129 peptide is known to bind weakly to DRB1*0401. This is in contrast to MBP81-99, for example, which binds with a high affinity to HLA-DR15.

Suitably, the TCR is capable of specifically binding to a peptide which has at least 90% identity to MBP 111-129 (SEQ ID NO: 3) or a fragment thereof when the peptide is presented by a major histocompatibility complex (MHC) molecule.

Suitably, the peptide may be capable of being presented by a HLA-DRB1*0401 molecule.

The TCR may comprise an α chain and a β chain,
wherein the α chain and the β chain each comprises three complementarity determining regions (CDRs) and the sequence of each CDR3 is as follows:
CDR3α-TVYGGATNKLIFGTGTLLAVQPNIQNPD (SEQ ID NO: 5)
CDR3β-SARGGSYNSPLHFGNGTRLTVTE (SEQ ID NO: 6)
   or a variant of those sequences having up to three amino acid changes.

The α chain of the TCR may comprise three CDRs having the following amino acid sequences:
CDR1α-TISGTDY (SEQ ID NO: 7)
CDR2α-GLTSN (SEQ ID NO: 8)
CDR3α-TVYGGATNKLIFGTGTLLAVQPNIQNPD (SEQ ID NO: 9)
   or variants of those sequences having up to three amino acid changes;
   and the β chain of the TCR may comprise three CDRs having the following amino acid sequences:
CDR1β-DFQATT (SEQ ID NO: 10)
CDR2β-SNEGSKA (SEQ ID NO: 11)
CDR3β-SARGGSYNSPLHFGNGTRLTVTE (SEQ ID NO: 12)
   or variants of those sequences having up to three amino acid changes.

The variable region of the α chain of the TCR may comprise an amino acid sequence having at least 80% sequence identity to SEQ ID NO:13, wherein the sequence identity does not include the CDR sequences; and the variable region of the β chain of the TCR may comprise an amino acid sequence having at least 80% sequence identity to SEQ ID NO: 14, wherein the sequence identity does not include the CDR sequences.

The variable region of the α chain of the TCR may comprise an amino acid sequence having at least 80% sequence identity to SEQ ID NO: 19; and the variable region of the β chain of the TCR may comprise an amino acid sequence having at least 80% sequence identity to SEQ ID NO: 21.

The constant region domains of the α chain and β chain of the TCR may each comprise an additional cysteine residue, enabling the formation of an extra disulphide bond between the a chain and the β chain. Suitably, the additional disulphide bond reduces mispairing with endogenous TCR chains.

The α chain of the TCR may comprise an amino acid sequence having at least 80% sequence identity to SEQ ID NO: 13; and the β chain of the TCR may comprise an amino acid sequence having at least 80% sequence identity to SEQ ID NO: 14.

In one aspect, the Treg is derived from a T cell isolated from a subject.

In another aspect, the present invention provides a pharmaceutical composition comprising an engineered Treg according to the invention.

In one aspect, the present invention relates to an engineered Treg or pharmaceutical composition according to the invention for use in treating a disease.

In another aspect, the present invention relates to the use of an engineered Treg or pharmaceutical composition according to the invention in the manufacture of a medicament.

In one aspect, there is provided a method for treating or preventing a disease in a subject in need of same which comprises the step of administering an engineered Treg or pharmaceutical composition according to the invention to the subject.

In another aspect, there is provided an engineered Treg or pharmaceutical composition for use, or a use or a method according to the invention, wherein the disease is multiple sclerosis.

In one aspect, there is provided an engineered Treg or pharmaceutical composition for use, or a use or a method according to the invention, wherein the subject is an HLADRB1*0401 positive subject.

In another aspect, there is provided a vector which comprises a nucleic acid sequence which encodes a TCR as defined herein and a nucleic acid sequence which encodes FOXP3.

In one aspect, a kit of polynucleotides or a kit of vectors is provided which comprises a first polynucleotide or vector which comprises a nucleic acid sequence which encodes a TCR as defined herein and a second polynucleotide or vector which comprises a nucleic acid sequence which encodes FOXP3. Suitably, the first and second polynucleotides or vectors are separate.

In one aspect, there is provided a method for producing an engineered Treg according to the invention which comprises the step of introducing into a cell in vitro or ex vivo a polynucleotide encoding a TCR as defined herein.

Suitably the T cell is a natural Treg which expresses FOXP3.

In one aspect, the method further comprises the step of introducing into the cell in vitro or ex vivo a polynucleotide encoding a FOXP3 protein.

Suitably the cell is a T cell.

Suitably the T cell is a ‘conventional’ T cell.

In one aspect of a method of the invention, the step of introducing the polynucleotide encoding a TCR and the polynucleotide encoding FOXP3 are performed sequentially, separately or simultaneously.

In another aspect of a method of the invention, the polynucleotide encoding a TCR and the polynucleotide encoding FOXP3 are introduced to the cell using the vector of the invention.

### DESCRIPTION OF THE FIGURES

FIG. 1—shows a schematic diagram of a pMP71 retroviral vector encoding (A) MBP TCR alpha and beta chains and (B) FOXP3 plus TCR alpha and beta chains.

FIG. 2—MS2-3C8 TCR pMP71. MS2-3C8 TCR recognises MBP 111-129 (SEQ ID NO: 3) presented by HLA-DRB1*0401. The TCR has been codon optimised and both constant alpha and beta domains have been murinised, and an extra disulphide bond has been added between c-alpha and c-beta.

FIG. 3—FOXP3-2A-MS2-3C8 TCR pMP71 comprising a FOXP3 gene with STOP codon removed at the 3′ T2A site, inserted into the RsrII and EcoR1 sites upstream of the MS-2 TCR.

FIG. 4—Schematic showing study design. A) Demonstration of MBP specific suppressive function in an engineered Treg. B) The use of an engineered Treg to suppress MS-like immunopathology in a HLA-transgenic mouse model

FIG. 5—Plots show representative flow cytometric analysis performed at day 7 to assess level of transduction through expression of murine TCR constant regions and FOXP3. Treg and Tconv cells are mock transduced, or are transduced with TCR (MS-2) or TCR+FOXP3 (MS-2 FOXP3).

FIG. 6—Graphs showing the relative expression of Treg surface markers on non transduced cells (d0) or gated on transduced cell populations (d7) n=2-4. These results demonstrate that regulatory T cells maintain FOXP3 expression during in vitro expansion.

FIG. 7—Graphs showing restimulation of effector T cells with peptide. Chinese Hamster Ovary (CHO) cells were transduced with human HLA-DR4 and CD80 or CD86. Cells expressing CD80 or CD86 were mixed together in equal parts for subsequent experiments. CHO cells were resuspended at 10×10⁶/mL in culture media with saturating amounts (10 μM/ml) of MBP 111-129 (SEQ ID NO: 3) (LSRFSWGAEGQRPGFGYGG). T cells transduced with a MS2 or MS2-FOXP3 construct were washed, counted and resuspended at 0.5×10⁶ cells/ml in complete RPMI. Cells were plated 1:1 with CHO cells incubated with or without peptide for 4 hours. Cells were fixed and permeablised before staining with antibodies for IL-2 and IFNγ. Transduction efficiency of T cells is indicated by ‘Td=’.

FIG. 8—Graphs showing restimulation of Treg cells with peptide. The method was described above for FIG. 7, using Tregs instead of effector T cells.

FIG. 9—Transduced T cells were cultured for 4 days with or without pepide-pulsed irradiated APC. Supernatant was collected and assayed for IL-2 and IFNg by ELISA (n=2-4) These data show that TCR-transduced Treg and TCR-FOXP3 converted Tconv are hyporesponsive to cognate peptide.

FIG. 10—shows IL-2 and IFNγ production from cells expressing the MS2 TCR and production from T cells expressing MS2 TCR and FOXP3 (n=3). Conventional T cells (T conv) transduced with TCR and TCR+FOXP3 produce less IL-2 than conventional cells transduced with TCR alone.

FIG. 11—TCR transduced T conv were stained with CFSE and cultured with or without peptide-pulsed irradiated APC at a ratio of 1 Tconv:0.01 APC for 4 days. Mock Treg (bar on the furthest left), MBP TCR-transduced Treg (second bar from the left), MBP TCR-FOXP3-transduced Treg (third bar from the left) and MBP TCR-FOXP3-transduced Tconv (fourth bar from the left in each group) were added in the indicated ratios. Proliferation was determined by analysing dilution of CFSE-stained Tconv (B). These data show that TCR-transduced Treg suppress T cell responses in an antigen-specific manner.

FIG. 12—TCR transduced T conv were stained with CFSE and cultured with or without peptide-pulsed irradiated APC at a ratio of 1 Tconv: 0.01 APC for 4 days. Mock Treg (bar on the furthest left), MBP TCR-transduced Treg (second bar from the left), MBP TCR-FOXP3-transduced Treg (third bar from the left) and MBP TCR-FOXP3-transduced Tconv (fourth bar from the left in each group) were added in the indicated ratios. Supernatants were collected and assayed for IL-2 by ELISA. These data show that TCR-transduced Treg suppress T cell responses in an antigen-specific manner.

FIG. 13—shows the sequence of SEQ ID NO: 18, the nucleotide sequence of the alpha chain V-region of MS2.3C8-alpha-variable (Va26-2) domain.

FIG. 14—shows the sequence of SEQ ID NO: 19, the amino acid sequence of the alpha chain V-region of MS2.3C8-alpha-variable (Va26-2) domain.

FIG. 15—shows the amino acid sequence of SEQ ID NO: 13, the human a V-region and murine constant-region.

FIG. 16—shows the sequence of SEQ ID NO: 20, the nucleotide sequence of the beta chain human V-region of the MS2.3C8-beta-variable domain.

FIG. 17—shows the amino acid sequence of SEQ ID NO: 21, the nucleotide sequence of the beta chain human V-region of the MS2.3C8-beta-variable domain.

FIG. 18—shows the sequence of SEQ ID NO: 14, the polypeptide sequence of the human β V-region and murine C-region.

FIG. 19—shows the sequence of SEQ ID NO: 22, MS2.3C8-alpha-variable amino acid sequence excluding CDR sequences.

FIG. 20—shows the sequence of SEQ ID NO: 23, MS2.3C8-beta-variable amino acid sequence excluding CDR sequences.

FIG. 21—shows the nucleic acid sequence of SEQ ID NO: 24, which encodes FOXP3.

FIG. 22—shows the amino acid sequence of SEQ ID NO: 25, FOXP3.

FIG. 23A-23C—CD4+CD25+ Treg were isolated from lymph nodes and splenocytes of HLA-DRB*0401 transgenic mice by bead sort. Treg were transduced with TCR+murine FOXP3. Transduced cells and equivalent numbers of CD45.1+OTI transgenic T cells were injected into HLA-DRB*0401 transgenic hosts conditioned with 4Gy irradiation. After 7 days mice were injected sub-cutaneously in the right or left flank with 30 ug ovalbumin (OVA) or 30 ug of OVA and 30 ug of human myelin basic protein (MBP) in incomplete Freud's adjuvant A. In FIG. 23A, paired FACS plots (a and b) show OTI cells identified by CD45.1 in the right and left inguinal lymph nodes of the same mouse. In paired FACS plots a, the left plot shows the data from an uninjected flank (no peptide) and the right shows data from the flank injected with OVA peptide B. In paired FACS plots b, the left plot shows the data from the flank injected with OVA and the right shows data from the flank injected with OVA+MBP peptides C. In paired panels c, cumulative data from 3 independent experiments showing % CD45.1+ cells (left panel) and absolute number of CD45.1 cells (right panel) in the inguinal lymph node of the flank that received OVA or OVA+MBP (n=9). In FIG. 23B, paired FACS plots d show data of transduced Treg that were positive for TCR staining at day 0 (left plot) and after 7 weeks (right plot). In FIG. 23C, graph e shows % CD45.1+ cells in the CD8+ population (left) and absolute number of CD45.1 cells (right panel) 7 weeks post-administration.

### DETAILED DESCRIPTION

### Myelin Basic Protein (MBP) Peptides

Myelin basic protein is important in the process of myelination of nerves and is found in the myelin sheath of cells in the nervous system such as oligodendrocytes and Schwann cells. MBP transcripts are also found in the bone marrow and the immune system. One function of the myelin sheath is to increase the velocity of axonal impulse conduction. MBP helps to maintain the correct structure of myelin and interacts with lipids in the myelin membrane. MBP is known to localise to the CNS and to various haematopoietic cells.

MBP has been implicated in the pathogenesis of demyelinating diseases, such as multiple sclerosis (MS). Studies have demonstrated a role for antibodies against MBP in the pathogenesis of MS.

In one aspect, an illustrative amino acid sequence of MBP comprises the sequence with UniProtKB accession P02686-1, shown as SEQ ID NO: 1:

(SEQ ID NO: 1)
MGNHAGKRELNAEKASTNSETNRGESEKKRNLGELSRTTSEDNEVFGEADA
NQNNGTSSQDTAVTDSKRTADPKNAWQDAHPADPGSRPHLIRLFSRDAPGR
EDNTFKDRPSESDELQTIQEDSAATSESLDVMASQKRPSQRHGSKYLATAS
TMDHARHGFLPRHRDTGILDSIGRFFGGDRGAPKRGSGKDSHHPARTAHYG
SLPQKSHGRTQDENPVVHFFKNIVTPRTPPPSQGKGRGLSLSRFSWGAEGQ
RPGFGYGGRASDYKSAHKGFKGVDAQGTLSKIFKLGGRDSRSGSPMARR.

In one aspect, an illustrative amino acid sequence of MBP comprises SEQ ID NO: 1 or a variant or fragment thereof.

In one aspect, the amino acid sequence of MBP comprises SEQ ID NO: 1.

In one aspect, the amino acid sequence of MBP is SEQ ID NO: 1.

Suitably, an illustrative amino acid sequence of MBP may be an isoform of UniProtKB accession P02686-1, such as UniProtKB accession P02686-5. Isoform P02686-5 differs from the canonical sequence shown above in SEQ ID NO:1 as follows, amino acid residues 1-133 are missing.

UniProtKB accession P02686-5 is shown as SEQ ID NO: 26:

(SEQ ID NO: 26)
MASQKRPSQRHGSKYLATASTMDHARHGFLPRHRDTGILDSIGRFFGGDRG
APKRGSGKDSHHPARTAHYGSLPQKSHGRTQDENPVVHFFKNIVTPRTPPP
SQGKGRGLSLSRFSWGAEGQRPGFGYGGRASDYKSAHKGFKGVDAQGTLSK
IFKLGGRDSRSGSPMARR.

In one aspect, the present invention provides an engineered Treg comprising a TCR which is capable of specifically binding to a peptide which comprises at least 90% identity to MBP 100-140: PPSQGKGRGLSLSRFSWGAEGQRPGFGYGGRASDYKSAHKG (SEQ ID NO: 2) or a fragment thereof when the peptide is presented by a major histocompatibility complex (MHC) molecule.

Suitably, the TCR is capable of specifically binding to a peptide which has at least 90% identity to MBP 100-140: PPSQGKGRGLSLSRFSWGAEGQRPGFGYGGRASDYKSAHKG (SEQ ID NO: 2) or a fragment thereof when the peptide is presented by a major histocompatibility complex (MHC) molecule.

Unless otherwise stated, MBP XXX-XXX as used herein refers to the numbering used in Muraro et al., JCI 1997; 100, 2, 339-349, incorporated herein by reference.

One may determine whether a peptide is capable of being presented by a MHC molecule and recognised by a T cell using methods available in the art. For example, an assay may comprise co-culturing antigen presenting cells (APCs) expressing the MHC: peptide complex to be tested with T cells comprising the TCR defined herein. T cell proliferation may then be measured as an indication of successful presentation of the peptide (for example by carboxyfluorescein succinimidyl ester (CFSE) assay). Alternatively, effector cytokine production may also be measured.

In one aspect, the MBP peptide comprises a sequence which comprises at least 90% identity to MBP 100-140: PPSQGKGRGLSLSRFSWGAEGQRPGFGYGGRASDYKSAHKG (SEQ ID NO: 2) or a fragment thereof. Suitably, the MBP peptide has at least 95%, 97%, 98% or 99% identity to MBP 100-140 (SEQ ID NO: 2) or a fragment thereof.

Suitably, the MBP peptide comprises a sequence which has at least 90% identity to MBP 100-140: PPSQGKGRGLSLSRFSWGAEGQRPGFGYGGRASDYKSAHKG (SEQ ID NO: 2) or a fragment thereof. Suitably, the MBP peptide has at least 95%, 97%, 98% or 99% identity to MBP 100-140 (SEQ ID NO: 2) or a fragment thereof.

The MBP peptide may be mutated compared to MBP 100-140 (SEQ ID NO: 2). For example, the MBP peptide may be mutated by amino acid insertion, deletion or substitution, so long as the modified MBP peptide retains the MHC binding specificity of the unmodified peptide, and is capable of being presented to a T cell. The MBP peptide may, for example have 3, 2, 1 or 0 mutations relative to MBP 100-140 (SEQ ID NO: 2). Suitably the MBP peptide may, for example have 3, 2, 1 or 0 conservative amino acid substitutions relative to MBP 100-140 (SEQ ID NO: 2). Suitably the MBP peptide may, for example have 3, 2, 1 or 0 insertions relative to

MBP 100-140 (SEQ ID NO: 2). Suitably the MBP peptide fragment may, for example have 3, 2, 1 or 0 deletions relative to MBP 100-140 (SEQ ID NO: 2).

As used herein “specifically binding” means that the TCR binds to the peptide but does not bind to other peptides, or binds at a lower affinity to other peptides.

The binding affinity between two molecules, e.g. a TCR and a peptide, or fragment thereof, may be quantified for example, by determination of the dissociation constant (KD). The KD can be determined by measurement of the kinetics of complex formation and dissociation between the TCR and the peptide, e.g. by the surface plas, on resonance (SPR) method (Biacore™). The rate constants corresponding to the association and the dissociation of a complex are referred to as the association rate constants ka (or kon) and dissociation rate constant kd. (or koff), respectively. KD is related to ka and kd through the equation KD=kd/ka.

Binding affinities associated with different molecular interactions, e.g. comparison of the binding affinity of different TCRs and peptides, may be compared by comparison of the KD values for the individual TCR/peptide complexes.

The peptide may be capable of being presented by any Human Leukocyte Antigen—antigen D Related (HLA-DR). For example, the peptide may be capable of being presented by a HLA-DR4, HLA-DR2, HLA-DR15, or HLA-DR16.

In one aspect, the peptide is capable of being presented by a HLA-DR4.

In one aspect, the peptide is capable of being presented by a HLA-DRB1*0401 molecule.

In one aspect, the peptide has at least 90% identity to MBP 111-129: LSRFSWGAEGQRPGFGYGG (SEQ ID NO:3). The MBP peptide may be mutated compared to MBP 111-129 (SEQ ID NO: 3). For example, the MBP peptide may be mutated by amino acid insertion, deletion or substitution, so long as the modified MBP peptide retains the MHC binding specificity of the unmodified peptide, and is capable of being presented to a T cell. The peptide may, for example have 3, 2, 1 or 0 mutations relative to MBP 111-129 (SEQ ID NO:

3). Suitably the peptide may, for example have 3, 2, 1 or 0 conservative mutations relative to MBP 111-129 (SEQ ID NO: 3). Suitably the peptide may, for example have 3, 2, 1 or 0 insertions relative to MBP 111-129 (SEQ ID NO: 3). Suitably the MBP peptide fragment may, for example have 3, 2, 1 or 0 deletions relative to MBP 111-129 (SEQ ID NO: 3). Suitably, the MBP 111-129 (SEQ ID NO: 3) peptide fragment retains the MHC binding specificity of the MBP 111-129 (SEQ ID NO: 3) peptide, and is capable of being presented to a T cell.

In one aspect, the peptide has at least 85% identity to MBP 116-123: WGAEGQRP (SEQ ID NO: 4).

In one aspect, the peptide has one, two or three amino acid substitutions, insertions or deletions compared to MBP 116-123: WGAEGQRP (SEQ ID NO: 4). Suitably the peptide has one, two or three conservative amino acid substitution compared to MBP 116-123: WGAEGQRP (SEQ ID NO: 4). Suitably the peptide has one, two or three insertions compared to MBP 116-123: WGAEGQRP (SEQ ID NO: 4). Suitably the MBP peptide fragment has one, two or three deletions compared to MBP 116-123: WGAEGQRP (SEQ ID NO: 4). Suitably, the MBP 116-123 (SEQ ID NO: 4) peptide fragment retains the MHC binding specificity of the MBP 116-123 (SEQ ID NO: 4) peptide, and is capable of being presented to a T cell.

In one aspect, the peptide comprises MBP 116-123: WGAEGQRP (SEQ ID NO: 4).

In one aspect, the peptide is MBP 116-123: WGAEGQRP (SEQ ID NO: 4).

### T Cell Receptor (TCR)

The variable domain of both the TCR a-chain and B-chain have three hypervariable or complementarity determining regions (CDRs). CDR3 is the main CDR responsible for recognizing processed antigen, although CDR1 of the alpha chain has also been shown to interact with the N-terminal part of the antigenic peptide, whereas CDR1 of the beta chain interacts with the C-terminal part of the peptide. CDR2 is thought to recognize the MHC molecule. Framework regions (FRs) are positioned between the CDRs. These regions provide the structure of the TCR variable region.

The TCR of the present invention comprises sufficient of the variable domains thereof to be able to interact with its peptide/MHC complex. Such interaction can be measured using a Biacore™ instrument, for example. Suitably the TCR may interact with HLADRB1*0401.

The repertoire of TCR variable regions is generated by combinatorial joining of variable (V), joining (J) and diversity (D) genes; and by N region diversification (nucleotides inserted by the enzyme deoxynucleotidyl-transferase).

α chains are formed from recombination events between the V and J segments. β chains are formed from recombination events involving the V, D and J segments.

The human TCRα locus, which also includes the TCRδ locus, is located on chromosome 14 (14q11.2). The TCRβ locus is located on chromosome 7 (7q34). The variable region of the TCRα chain is formed by recombination between one of 46 different Vα (variable) segments and one of 58 Jα (joining) segments (Koop et al.; 1994; Genomics; 19:478-493 incorporated herein by reference). The variable region of a TCRβ chain is formed from recombination between 54 Vβ, 14 Jβand 2 Dβ (diversity) segments (Rowen et al.; 1996; Science; 272:1755-1762 incorporated herein by reference).

The V and J (and D as appropriate) gene segments for each TCR chain locus have been identified and the germline sequence of each gene is known and annotated (for example see Scaviner & Lefranc; 2000; Exp Clin Immunogenet; 17:83-96 and Folch & Lefranc; 2000; Exp Clin Immunogenet; 17:42-54, incorporated herein by reference).

FR1, CDR1, FR2, CDR2, FR3 and CDR3 of the α chain of natural TCRs are encoded by the Va gene. FR1, CDR1, FR2, CDR2 and FR3 of the β chain of natural TCRs are encoded by the Vβ gene.

As the germline sequence of each variable gene is known in the art (see Scaviner & Lefranc; as above and Folch & Lefranc; supra) the Va and/or VB of a particular TCR can be sequenced and the germline V segment which is utilised in the TCR can be identified (see, for example, Hodges et al.; 2003; J Clin Pathol; 56:1-11, Zhou et al.; 2006; Laboratory Investigation; 86; 314-321, incorporated herein by reference).

The present invention provides an engineered Treg comprising an engineered T cell receptor.

In one aspect, the invention provides an engineered Treg comprising a TCR which is capable of specifically binding to a peptide which comprises at least 90% identity to MBP 100-140 (SEQ ID NO: 2) or a fragment thereof when the peptide is presented by a major histocompatibility complex (MHC) molecule.

Suitably, the invention provides an engineered Treg comprising a TCR which is capable of specifically binding to a peptide which has at least 90% identity to MBP 100-140 (SEQ ID NO: 2) or a fragment thereof when the peptide is presented by a major histocompatibility complex (MHC) molecule.

In one aspect, the TCR comprises an α chain and a β chain,
wherein the α chain and the β chain each comprises three complementarity determining regions (CDRs) and the sequence of each CDR3 is as follows:

CDR3α
(SEQ ID NO: 5)
TVYGGATNKLIFGTGTLLAVQPNIQNPD
CDR3β
(SEQ ID NO: 6)
SARGGSYNSPLHFGNGTRLTVTE

or a variant of those sequences having up to three amino acid changes.

In one aspect, the α chain of the TCR comprises three CDRs having the following amino acid sequences:

CDRlα
(SEQ ID NO: 7)
TISGTDY
CDR2α
(SEQ ID NO: 8)
GLTSN
CDR3α
(SEQ ID NO: 9)
TVYGGATNKLIFGTGTLLAVQPNIQNPD

or variants of those sequences having up to three amino acid changes; and wherein the β chain of the TCR comprises three CDRs having the following amino acid sequences:

CDR1β
(SEQ ID NO: 10)
DFQATT
CDR2β
(SEQ ID NO: 11)
SNEGSKA
CDR3β
(SEQ ID NO: 12)
SARGGSYNSPLHFGNGTRLTVTE

or variants of those sequences having up to three amino acid changes.

Suitably the amino acid change in a CDR is a conservative substitution, insertion or deletion. Preferably the amino acid change is a conservative substitution.

In one aspect, the variable region of the α chain of the TCR comprises an amino acid sequence having at least 80% sequence identity to SEQ ID NO:19, and the variable region of the β chain of the TCR comprises an amino acid sequence having at least 80% sequence identity to SEQ ID NO: 21, wherein the sequence identity does not include the CDR sequences. Suitably the CDR sequences are as disclosed herein. Suitably, the variable region of the α chain of the TCR comprises an amino acid sequence having at least 80%, 85%, 90%, 95% or 97% sequence identity to SEQ ID NO:19, and the variable region of the β chain of the TCR comprises an amino acid sequence having at least 80%, 85%, 90%, 95%, or 97% sequence identity to SEQ ID NO: 21.

Suitably, the variable region of the α chain of the TCR comprises an amino acid sequence may have at least 85% sequence identity to SEQ ID NO:19, and the variable region of the β chain of the TCR comprises an amino acid sequence having at least 85% sequence identity to SEQ

ID NO: 21, wherein the sequence identity does not include the CDR sequences. Suitably, the variable region of the α chain of the TCR comprises an amino acid sequence may have at least 90% sequence identity to SEQ ID NO: 19, and the variable region of the β chain of the TCR comprises an amino acid sequence having at least 90% sequence identity to SEQ ID NO: 21, wherein the sequence identity does not include the CDR sequences. Suitably, the variable region of the α chain of the TCR comprises an amino acid sequence may have at least 95% sequence identity to SEQ ID NO:19 and the variable region of the β chain of the TCR comprises an amino acid sequence having at least 95% sequence identity to SEQ ID NO: 21, wherein the sequence identity does not include the CDR sequences. Suitably, the variable region of the α chain of the TCR comprises an amino acid sequence may have at least 97% sequence identity to SEQ ID NO:19 and the variable region of the β chain of the TCR comprises an amino acid sequence having at least 97% sequence identity to SEQ ID NO: 21, wherein the sequence identity does not include the CDR sequences. Suitably, the variable region of the α chain of the TCR comprises an amino acid sequence set fort in SEQ ID NO: 19 and the variable region of the β chain of the TCR comprises an amino acid sequence set forth in SEQ ID NO: 21, wherein the sequence identity does not include the CDR sequences.

In another aspect, the variable region of the α chain of the TCR comprises an amino acid sequence having at least 80% sequence identity to SEQ ID NO: 19; and the variable region of the β chain of the TCR comprises an amino acid sequence having at least 80% sequence identity to SEQ ID NO: 21.

Suitably, the variable region of the α chain of the TCR comprises an amino acid sequence having at least 85% sequence identity to SEQ ID NO: 19; and the variable region of the β chain of the TCR comprises an amino acid sequence having at least 85% sequence identity to SEQ ID NO: 21. Suitably, the variable region of the α chain of the TCR comprises an amino acid sequence having at least 90% sequence identity to SEQ ID NO: 19; and the variable region of the β chain of the TCR comprises an amino acid sequence having at least 90% sequence identity to SEQ ID NO: 21. Suitably, the variable region of the α chain of the TCR comprises an amino acid sequence having at least 95% sequence identity to SEQ ID NO: 19; and the variable region of the β chain of the TCR comprises an amino acid sequence having at least 95% sequence identity to SEQ ID NO: 21. Suitably, the variable region of the α chain of the TCR comprises an amino acid sequence having at least 97% sequence identity to SEQ ID NO: 19; and the variable region of the β chain of the TCR comprises an amino acid sequence having at least 97% sequence identity to SEQ ID NO: 21.

In one aspect, the α chain of the TCR comprises an amino acid sequence having at least 80% sequence identity to SEQ ID NO: 13; and the β chain of the TCR comprises an amino acid sequence having at least 80% sequence identity to SEQ ID NO: 14.

Suitably, the α chain of the TCR comprises an amino acid sequence having at least 85% sequence identity to SEQ ID NO: 13; and the β chain of the TCR comprises an amino acid sequence having at least 85% sequence identity to SEQ ID NO: 14. Suitably, the α chain of the TCR comprises an amino acid sequence having at least 90% sequence identity to SEQ ID NO: 13; and the β chain of the TCR comprises an amino acid sequence having at least 90% sequence identity to SEQ ID NO: 14. Suitably, the α chain of the TCR comprises an amino acid sequence having at least 95% sequence identity to SEQ ID NO: 13; and the β chain of the TCR comprises an amino acid sequence having at least 95% sequence identity to SEQ ID NO: 14. Suitably, the α chain of the TCR comprises an amino acid sequence having at least 97% sequence identity to SEQ ID NO: 13; and the β chain of the TCR comprises an amino acid sequence having at least 97% sequence identity to SEQ ID NO: 14.

In another aspect, the constant region domains of the α chain and β chain of the TCR each comprise an additional cysteine residue, enabling the formation of an extra disulphide bond between the α chain and the β chain.

Suitably, residue 48 in the constant alpha chain is converted from a threonine to a cysteine and residue 57 of the constant beta chain is converted from a serine to a cysteine for the formation of the additional disulphide bond.

Suitably, the TCR is codon optimised.

Suitably, the TCR is codon optimised for expression in a mouse.

In one aspect the constant domains employed in the TCR are murine sequences.

Suitably the constant regions have been murinised. For example, both the constant-alpha and the constant-beta domains have been murinised.

In another aspect, the TCR is codon optimised for expression in a human. Suitably, the constant domains employed in the TCR are human sequences.

In one aspect the TCR may comprise, for example, human variable regions and murine constant regions.

The present TCR may comprise one or more amino acid residues as defined herein which is not encoded by the germline Vα or Vβ gene. In other words, the TCR may comprise part of an α chain and/or β chain which comprises an altered amino acid residue at one or more of the positions described herein, compared to the corresponding α chain and/or β chain as encoded by the unaltered germline Vα or Vβ gene.

The amino acid residues identified herein as framework (FR) or complementarity-determining regions (CDRs) are identified according to the International ImMunoGeneTics information system' (IMGT). This system is well known in the art (Lefrance et al.; 2003; Dev Comp Immunol; 27:55-77) and is based on the high conservation of the structure of the variable region. The numbering takes into account and combines the definition of the FR and CDRs, structural data from X-ray diffraction studies and the characterization of the hypervariable loops.

The delimitations of the FR and CDR regions are defined within the IMGT numbering system. The FR1 region comprises positions 1-26 (25-26 amino acids, depending on the V-GENE group or subgroup) with 1st-CYS at position 23. The FR2 region comprises positions 39-55 (16-17 amino acids) with a conserved TRP at position 41. The FR3 region comprises positions 66-104 (36-39 amino acids, depending on the VGENE group or subgroup) with a conserved hydrophobic amino acid at position 89 and the 2nd-CYS at position 104. Residue 1 of the IGMT numbering system is the first residue in FR1. Residue 104 of the IGMT numbering system is the last residue in FR3.

Methods suitable for generating a TCR according to the present invention are known in the art.

For example mutagenesis may be performed to alter specific nucleotides in a nucleic acid sequence encoding the TCR. Such mutagenesis will alter the amino acid sequence of the TCR so that it comprises one or more of the amino acid residues as described herein.

An example of a mutagenesis method is the Quikchange method (Papworth et al.; 1996; Strategies; 9 (3); 3-4). This method involves the use of a pair of complementary mutagenic primers to amplify a template nucleic acid sequence in a thermocycling reaction using a high-fidelity non-strand-displacing DNA polymerase, such as pfu polymerase.

The terms “one or more” or “at least one” as used herein may include one, two, three, four, five, six, seven, eight, nine, ten, eleven, twelve, thirteen, fourteen, fifteen, sixteen, seventeen, eighteen, nineteen, twenty or more amino acid residues as described herein.

The term “two or more” as used herein may include two, three, four, five, six, seven, eight, nine, ten, eleven, twelve, thirteen, fourteen, fifteen, sixteen, seventeen, eighteen, nineteen, twenty or more amino acid residues as described herein.

### Conservative Substitution

Suitably, the amino acid residues present at a given position in the present invention may be defined as a residue which is biochemically similar to the amino acids recited for the given SEQ ID NOS.

Amino acids with similar biochemical properties may be defined as amino acids which can be substituted via a conservative substitution.

Conservative amino acid substitutions may be made on the basis of similarity in polarity, charge, solubility, hydrophobicity, hydrophilicity, and/or the amphipathic nature of the residues as long as high expression of the TCR is retained. For example, negatively charged amino acids include aspartic acid and glutamic acid; positively charged amino acids include lysine and arginine; and amino acids with uncharged polar head groups having similar hydrophilicity values include leucine, isoleucine, valine, glycine, alanine, asparagine, glutamine, serine, threonine, phenylalanine, and tyrosine.

Conservative substitutions may be made, for example according to Table 3 below. Amino acids in the same block in the second column and preferably in the same line in the third column may be substituted for each other:

TABLE 3

| | | |
|---|---|---|
| ALIPHATIC | Non-polar | G A P |
| | | I L V |
| | Polar - uncharged | C S T M |
| | | N Q |
| | Polar - charged | D E |
| | | K R |
| AROMATIC | | H F W Y |

The present invention also encompasses homologous substitution (substitution and replacement are both used herein to mean the interchange of an existing amino acid residue, with an alternative residue) i.e. like-for-like substitution such as basic for basic, acidic for acidic, polar for polar etc.

Unless otherwise explicitly stated herein by way of reference to a specific, individual amino acid, amino acids may be substituted using conservative substitutions as recited below.

An aliphatic, non-polar amino acid may be a glycine, alanine, proline, isoleucine, leucine or valine residue.

An aliphatic, polar uncharged amino may be a cysteine, serine, threonine, methionine, asparagine or glutamine residue.

An aliphatic, polar charged amino acid may be an aspartic acid, glutamic acid, lysine or arginine residue.

An aromatic amino acid may be a histidine, phenylalanine, tryptophan or tyrosine residue.

Suitably, a conservative substitution may be made between amino acids in the same line in Table 3.

### Sequences

The present invention further provides a nucleotide sequence encoding a TCR α chain and/or β chain described herein. In one aspect, a nucleotide sequence encoding a TCR described herein may be introduced into a cell.

As used herein, the term “introduced” refers to methods for inserting foreign DNA into a cell. As used herein the term introduced includes both transduction and transfection methods. Transfection is the process of introducing nucleic acids into a cell by non-viral methods. Transduction is the process of introducing foreign DNA into a cell via a viral vector.

As used herein, the terms “polynucleotide” and “nucleic acid” are intended to be synonymous with each other. The nucleic acid sequence may be any suitable type of nucleotide sequence, such as a synthetic RNA/DNA sequence, a cDNA sequence or a partial genomic DNA sequence.

The term “polypeptide” as used herein is used in the normal sense to mean a series of residues, typically L-amino acids, connected one to the other typically by peptide bonds between the α-amino and carboxyl groups of adjacent amino acids. The term is synonymous with “protein”.

It will be understood by a skilled person that numerous different polynucleotides and nucleic acids can encode the same polypeptide as a result of the degeneracy of the genetic code. In addition, it is to be understood that skilled persons may, using routine techniques, make nucleotide substitutions that do not affect the polypeptide sequence encoded by the polynucleotides described here to reflect the codon usage of any particular host organism in which the polypeptides are to be expressed.

Nucleic acids according to the invention may comprise DNA or RNA. They may be single-stranded or double-stranded. They may also be polynucleotides which include within them synthetic or modified nucleotides. A number of different types of modification to oligonucleotides are known in the art. These include methylphosphonate and phosphorothioate backbones, addition of acridine or polylysine chains at the 3′ and/or 5′ ends of the molecule. For the purposes of the use as described herein, it is to be understood that the polynucleotides may be modified by any method available in the art. Such modifications may be carried out in order to enhance the in vivo activity or life span of polynucleotides of interest.

The polynucleotide may be in isolated or recombinant form. It may be incorporated into a vector and the vector may be incorporated into a host cell. Such vectors and suitable hosts form yet further aspects of the present invention.

The polynucleotide may be double or single stranded, and may be RNA or DNA.

The polynucleotide may be codon optimised. Different cells differ in their usage of particular codons. This codon bias corresponds to a bias in the relative abundance of particular tRNAs in the cell type. By altering the codons in the sequence so that they are tailored to match with the relative abundance of corresponding tRNAs, it is possible to increase expression. Suitably the polynucleotide may be codon optimised for expression in a murine model of disease. Suitably, the polynucleotide may be codon optimised for expression in a human subject.

Many viruses, including HIV and other lentiviruses, use a large number of rare codons and by changing these to correspond to commonly used mammalian codons, increased expression of the packaging components in mammalian producer cells can be achieved. Codon usage tables are known in the art for mammalian cells, as well as for a variety of other organisms.

Codon optimisation may also involve the removal of mRNA instability motifs and cryptic splice sites.

The polynucleotide may comprise a nucleic acid sequence which enables both a nucleic acid sequence encoding an α chain and a nucleic acid sequence a β chain to be expressed from the same mRNA transcript.

For example, the polynucleotide may comprise an internal ribosome entry site (IRES) between the nucleic acid sequences which encode the α chain and the β chain. An IRES is a nucleotide sequence that allows for translation initiation in the middle of a mRNA sequence.

The polynucleotide may comprise a nucleic acid sequence encoding an α chain and a nucleic acid sequence a β chain linked by an internal self-cleaving sequence.

The internal self-cleaving sequence may be any sequence which enables the polypeptide comprising the α chain and the polypeptide comprising the β chain to become separated.

The cleavage site may be self-cleaving, such that when the polypeptide is produced, it is immediately cleaved into individual peptides without the need for any external cleavage activity.

The term “cleavage” is used herein for convenience, but the cleavage site may cause the peptides to separate into individual entities by a mechanism other than classical cleavage. For example, for the Foot-and-Mouth disease virus (FMDV) 2A self-cleaving peptide, various models have been proposed for to account for the “cleavage” activity: proteolysis by a host-cell proteinase, autoproteolysis or a translational effect (Donnelly et al (2001) J. Gen. Virol. 82:1027-1041 incorporated herein by reference). The exact mechanism of such “cleavage” is not important for the purposes of the present invention, as long as the cleavage site, when positioned between nucleic acid sequences which encode proteins, causes the proteins to be expressed as separate entities.

The self-cleaving peptide may be a 2A self-cleaving peptide from an aphtho-or a cardiovirus.

A variant can be considered in terms of similarity (i.e. amino acid residues having similar chemical properties/functions), preferably a variant is expressed in terms of sequence identity.

Sequence comparisons can be conducted by eye, or more usually, with the aid of readily available sequence comparison programs. These publicly and commercially available computer programs can calculate sequence identity between two or more sequences.

Sequence identity may be calculated over contiguous sequences, i.e. one sequence is aligned with the other sequence and each amino acid in one sequence directly compared with the corresponding amino acid in the other sequence, one residue at a time. This is called an “ungapped” alignment. Typically, such ungapped alignments are performed only over a relatively short number of residues (for example less than 50 contiguous amino acids).

Although this is a very simple and consistent method, it fails to take into consideration that, for example, in an otherwise identical pair of sequences, one insertion or deletion will cause the following amino acid residues to be put out of alignment, thus potentially resulting in a large reduction in % homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible—reflecting higher relatedness between the two compared sequences—will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example when using the GCG Wisconsin Bestfit package (see below) the default gap penalty for amino acid sequences is −12 for a gap and −4 for each extension.

Calculation of maximum % sequence identity therefore firstly requires the production of an optimal alignment, taking into consideration gap penalties. A suitable computer program for carrying out such an alignment is the GCG Wisconsin Bestfit package (University of Wisconsin, U.S.A; Devereux et al., 1984, Nucleic Acids Research 12:387 incorporated herein by reference). Examples of other software than can perform sequence comparisons include, but are not limited to, the BLAST package (see Ausubel et al., 1999 ibid—Chapter 18), FASTA (Atschul et al., 1990, J. Mol. Biol., 403-410 incorporated herein by reference) and the GENEWORKS suite of comparison tools. Both BLAST and FASTA are available for offline and online searching (see Ausubel et al., 1999 ibid, pages 7-58 to 7-60 incorporated herein by reference). However it is preferred to use the GCG Bestfit program.

In one embodiment, the sequence identity is determined across the entirety of the sequence. In one embodiment, the sequence identity is determined across the entirety of the candidate sequence being compared to a sequence recited herein.

Although the final sequence identity can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix-the default matrix for the BLAST suite of programs. GCG

Wisconsin programs generally use either the public default values or a custom symbol comparison table if supplied (see user manual for further details). It is preferred to use the public default values for the GCG package, or in the case of other software, the default matrix, such as BLOSUM62.

Once the software has produced an optimal alignment, it is possible to calculate % sequence identity. The software typically does this as part of the sequence comparison and generates a numerical result.

The term “variant” according to the present invention includes any substitution of, variation of, modification of, replacement of, deletion of or addition of one (or more) amino acids from or to the sequence providing the resultant amino acid sequence retains substantially the same activity as the unmodified sequence. For example, conservative amino acid substitutions may be made. As used herein, a variant polypeptide is taken to include a polypeptide comprising an amino acid sequence which is at least 70, 80, 85, 90, 95, 98 or 99% identical to a sequence shown herein.

In one aspect, the variant maintains the function of the parent sequence.

In one aspect, a cell according to the invention a nucleotide sequence which encodes a FOXP3 protein has also been introduced to the cell.

In one aspect, the cell, engineered Treg or pharmaceutical composition of the present invention may comprise a nucleic acid sequence which encodes a FOXP3 protein, suitably the nucleic acid sequence encodes an amino acid sequence shown as SEQ ID NO: 17 or an amino acid sequence which is at least 80, 85, 90, 95, 98 or 99% identical, preferably at least 95 or 99% identical to a sequence shown herein as SEQ ID NO: 17.

The nucleic acid encoding the TCR and/or FOXP3 may comprise a leader sequence upstream of the initiation codon. This sequence may regulate translation of a transcript. By ay of example, suitable leader sequences for use in the present invention are: MKLVTSITVLLSLGIMG (SEQ ID NO: 15) and MLLLLLLLGPGISLLLPGSLAGSGL (SEQ ID NO: 16).

In a further aspect the present invention provides a kit of nucleic acid sequences comprising: a first nucleic acid sequence which encodes a TCR as defined herein and a second nucleic acid which encodes FOXP3.

### Vector

The present invention also provides a vector comprising a nucleotide sequence encoding a TCR as described herein. Suitably, the vector may additionally comprise a nucleotide sequence encoding a forkhead box P3 (FOXP3) polypeptide. In one aspect, there is provided a kit of vectors which comprises one or more nucleic acid sequence(s) of the invention such as a nucleic acid encoding a TCR as defined herein and a nucleic acid encoding FOXP3.

FOXP3 is a member of the FOX protein family of transcription factors and functions as a master regulator of the regulatory pathway in the development and function of regulatory T cells. Suitably, the FOXP3 polypeptide is from a human e.g. the UniProtKB accession:

Q9BZS1:
(SEQ ID NO: 17)
MPNPRPGKPSAPSLALGPSPGASPSWRAAPKASDLLGARGPGGTFQGRDLR
GGAHASSSSLNPMPPSQLQLPTLPLVMVAPSGARLGPLPHLQALLQDRPHF
MHQLSTVDAHARTPVLQVHPLESPAMISLTPPTTATGVFSLKARPGLPPGI
NVASLEWVSREPALLCTFPNPSAPRKDSTLSAVPQSSYPLLANGVCKWPGC
EKVFEEPEDFLKHCQADHLLDEKGRAQCLLQREMVQSLEQQLVLEKEKLSA
MQAHLAGKMALTKASSVASSDKGSCCIVAAGSQGPVVPAWSGPREAPDSLF
AVRRHLWGSHGNSTFPEFLHNMDYFKFHNMRPPFTYATLIRWAILEAPEKQ
RTLNEIYHWFTRMFAFFRNHPATWKNAIRHNLSLHKCFVRVESEKGAVWTV
DELEFRKKRSQRPSRCSNPTPGP.

Suitably, the FOXP3 polypeptide comprises an amino acid sequence set forth in SEQ ID NO: 17, or a fragment thereof. Suitably the FOXP3 polypeptide comprises an amino acid sequence which is at least 80% identical to SEQ ID NO: 17 or a fragment thereof. Suitably, the polypeptide comprises an amino acid sequence which is 85, 90, 95, 98 or 99% identical to SEQ ID NO: 17 or a fragment thereof. Suitably the fragment retains FOXP3 activity. Suitably the fragment is able to bind to FOXP3 targets and act as a transcription factor.

Suitably, the FOXP3 polypeptide may be a natural variant of SEQ ID NO: 17. Suitably, the FOXP3 polypeptide is an isoform of SEQ ID NO: 17. For example, the FOXP3 polypeptide may comprise a deletion of amino acid positions 72-106 relative to SEQ ID NO: 17. Alternatively, the FOXP3 polypeptide may comprise a deletion of amino acid positions 246-272 relative to SEQ ID NO: 17.

Suitably, the FOXP3 polypeptide comprises the amino acid sequence set forth in SEQ ID NO: 25:

(SEQ ID NO: 25)
MPNPRPGKPSAPSLALGPSPGASPSWRAAPKASDLLGARGPGGTFQGRDLR
GGAHASSSSLNPMPPSQLQLPTLPLVMVAPSGARLGPLPHLQALLQDRPHF
MHQLSTVDAHARTPVLQVHPLESPAMISLTPPTTATGVFSLKARPGLPPGI
NVASLEWVSREPALLCTFPNPSAPRKDSTLSAVPQSSYPLLANGVCKWPGC
EKVFEEPEDFLKHCQADHLLDEKGRAQCLLQREMVQSLEQVEELSAMQAHL
AGKMALTKASSVASSDKGSCCIVAAGSQGPVVPAWSGPREAPDSLFAVRRH
LWGSHGNSTFPEFLHNMDYFKFHNMRPPFTYATLIRWAILEAPEKQRTLNE
IYHWFTRMFAFFRNHPATWKNAIRHNLSLHKCFVRVESEKGAVWTVDELEF
RKKRSQRPSRCSNPTPGPEGRGSLLTCGDVEEN.

Suitably, the FOXP3 polypeptide comprises an amino acid sequence set forth in SEQ ID NO: 25, or a fragment thereof. Suitably the FOXP3 polypeptide comprises an amino acid sequence which is at least 80% identical to SEQ ID NO: 25 or a fragment thereof. Suitably, the polypeptide comprises an amino acid sequence which is 85, 90, 95, 98 or 99% identical to

SEQ ID NO: 25 or a fragment thereof. Suitably the fragment retains FOXP3 activity. Suitably the fragment is able to bind to FOXP3 targets and act as a transcription factor.

Suitably, the FOXP3 polypeptide may be a natural variant of SEQ ID NO: 25. Suitably, the FOXP3 polypeptide is an isoform of SEQ ID NO: 25. For example, the FOXP3 polypeptide may comprise a deletion of amino acid positions 72-106 relative to SEQ ID NO: 25.

Alternatively, the FOXP3 polypeptide may comprise a deletion of amino acid positions 246-272 relative to SEQ ID NO: 25.

Suitably, the FOXP3 polypeptide is encoded by the polynucleotide sequence set forth in SEQ ID NO: 29:

(SEQ ID NO: 27)
ATGCCCAACCCCAGGCCTGGCAAGCCCTCGGCCCCTTCCTTGGCCCTTGGC
CCATCCCCAGGAGCCTCGCCCAGCTGGAGGGCTGCACCCAAAGCCTCAGAC
CTGCTGGGGGCCCGGGGCCCAGGGGGAACCTTCCAGGGCCGAGATCTTCGA
GGCGGGGCCCATGCCTCCTCTTCTTCCTTGAACCCCATGCCACCATCGCAG
CTGCAGCTGCCCACACTGCCCCTAGTCATGGTGGCACCCTCCGGGGCACGG
CTGGGCCCCTTGCCCCACTTACAGGCACTCCTCCAGGACAGGCCACATTTC
ATGCACCAGCTCTCAACGGTGGATGCCCACGCCCGGACCCCTGTGCTGCAG
GTGCACCCCCTGGAGAGCCCAGCCATGATCAGCCTCACACCACCCACCACC
GCCACTGGGGTCTTCTCCCTCAAGGCCCGGCCTGGCCTCCCACCTGGGATC
AACGTGGCCAGCCTGGAATGGGTGTCCAGGGAGCCGGCACTGCTCTGCACC
TTCCCAAATCCCAGTGCACCCAGGAAGGACAGCACCCTTTCGGCTGTGCCC
CAGAGCTCCTACCCACTGCTGGCAAATGGTGTCTGCAAGTGGCCCGGATGT
GAGAAGGTCTTCGAAGAGCCAGAGGACTTCCTCAAGCACTGCCAGGCGGAC
CATCTTCTGGATGAGAAGGGCAGGGCACAATGTCTCCTCCAGAGAGAGATG
GTACAGTCTCTGGAGCAGCAGCTGGTGCTGGAGAAGGAGAAGCTGAGTGCC
ATGCAGGCCCACCTGGCTGGGAAAATGGCACTGACCAAGGCTTCATCTGTG
GCATCATCCGACAAGGGCTCCTGCTGCATCGTAGCTGCTGGCAGCCAAGGC
CCTGTCGTCCCAGCCTGGTCTGGCCCCCGGGAGGCCCCTGACAGCCTGTTT
GCTGTCCGGAGGCACCTGTGGGGTAGCCATGGAAACAGCACATTCCCAGAG
TTCCTCCACAACATGGACTACTTCAAGTTCCACAACATGCGACCCCCTTTC
ACCTACGCCACGCTCATCCGCTGGGCCATCCTGGAGGCTCCAGAGAAGCAG
CGGACACTCAATGAGATCTACCACTGGTTCACACGCATGTTTGCCTTCTTC
AGAAACCATCCTGCCACCTGGAAGAACGCCATCCGCCACAACCTGAGTCTG
CACAAGTGCTTTGTGCGGGTGGAGAGCGAGAAGGGGGCTGTGTGGACCGTG
GATGAGCTGGAGTTCCGCAAGAAACGGAGCCAGAGGCCCAGCAGGTGTTCC
AACCCTACACCTGGCCCCTGA

In some embodiments of the invention, the polynucleotide encoding the FOXP3 polypeptide or variant comprises a polynucleotide sequence which is at least 80% identical to SEQ ID NO:

27 or a functional fragment thereof. Suitably, the polynucleotide encoding the FOXP3 polypeptide or variant comprises a polynucleotide sequence which is at least 85, 90, 95, 98 or 99% identical to SEQ ID NO: 27 or a functional fragment thereof. In some embodiments of the invention, the polynucleotide encoding the FOXP3 polypeptide or variant comprises SEQ ID NO: 27 or a functional fragment thereof.

Suitably, the FOXP3 polypeptide is encoded by the nucleic acid sequence set forth in SEQ ID NO: 24:

(SEQ ID NO: 24)
GAATTCGTCGACATGCCCAACCCCAGACCCGGCAAGCCTTCTGCCCCTTCT
CTGGCCCTGGGACCATCTCCTGGCGCCTCCCCATCTTGGAGAGCCGCCCCT
AAAGCCAGCGATCTGCTGGGAGCTAGAGGCCCTGGCGGCACATTCCAGGGC
AGAGATCTGAGAGGCGGAGCCCACGCCTCTAGCAGCAGCCTGAATCCCATG
CCCCCTAGCCAGCTGCAGCTGCCTACACTGCCTCTCGTGATGGTGGCCCCT
AGCGGAGCTAGACTGGGCCCTCTGCCTCATCTGCAGGCTCTGCTGCAGGAC
CGGCCCCACTTTATGCACCAGCTGAGCACCGTGGACGCCCACGCCAGAACA
CCTGTGCTGCAGGTGCACCCCCTGGAAAGCCCTGCCATGATCAGCCTGACC
CCTCCAACCACAGCCACCGGCGTGTTCAGCCTGAAGGCCAGACCTGGACTG
CCCCCTGGCATCAATGTGGCCAGCCTGGAATGGGTGTCCCGCGAACCTGCC
CTGCTGTGCACCTTCCCCAATCCTAGCGCCCCCAGAAAGGACAGCACACTG
TCTGCCGTGCCCCAGAGCAGCTATCCCCTGCTGGCTAACGGCGTGTGCAAG
TGGCCTGGCTGCGAGAAGGTGTTCGAGGAACCCGAGGACTTCCTGAAGCAC
TGCCAGGCCGACCATCTGCTGGACGAGAAAGGCAGAGCCCAGTGCCTGCTG
CAGCGCGAGATGGTGCAGTCCCTGGAACAGCAGCTGGTGCTGGAAAAAGAA
AAGCTGAGCGCCATGCAGGCCCACCTGGCCGGAAAGATGGCCCTGACAAAA
GCCAGCAGCGTGGCCAGCTCCGACAAGGGCAGCTGTTGTATCGTGGCCGCT
GGCAGCCAGGGACCTGTGGTGCCTGCTTGGAGCGGACCTAGAGAGGCCCCC
GATAGCCTGTTTGCCGTGCGGAGACACCTGTGGGGCAGCCACGGCAACTCT
ACCTTCCCCGAGTTCCTGCACAACATGGACTACTTCAAGTTCCACAACATG
AGGCCCCCCTTCACCTACGCCACCCTGATCAGATGGGCCATTCTGGAAGCC
CCCGAGAAGCAGCGGACCCTGAACGAGATCTACCACTGGTTTACCCGGATG
TTCGCCTTCTTCCGGAACCACCCCGCCACCTGGAAGAACGCCATCCGGCAC
AATCTGAGCCTGCACAAGTGCTTCGTGCGGGTGGAAAGCGAGAAGGGCGCC
GTGTGGACAGTGGACGAGCTGGAATTTCGGAAGAAGCGGTCCCAGAGGCCC
AGCCGGTGTAGCAATCCTACACCTGGCCCTGAGGGCAGAGGAAGTCTGCTA
ACATGCGGTGACGTCGAGGAGAATCC.

Suitably, the FOXP3 polypeptide is encoded by the nucleic acid sequence set forth in SEQ ID NO: 24, or a fragment thereof. Suitably the FOXP3 polypeptide is encoded by a nucleic acid sequence which is at least 80% identical to SEQ ID NO: 24 or a fragment thereof. Suitably, the FOXP3 polypeptide is encoded by the nucleic acid sequence which is 85, 90, 95, 98 or 99% identical to SEQ ID NO: 24 or a fragment thereof. Suitably the fragment retains FOXP3 activity.

Suitably the polypeptide encoded by the fragment is able to bind to FOXP3 targets and act as a transcription factor.

The term “vector” includes an expression vector, i.e., a construct enabling expression of TCR i.e. an α chain and/or β chain according to the present invention. Suitably the expression vector additionally enables expression of a FOXP3 polypeptide. In some embodiments, the vector is a cloning vector.

Suitable vectors may include, but are not limited to, plasmids, viral vectors, transposons, nucleic acid complexed with polypeptide or immobilised onto a solid phase particle.

Viral delivery systems include but are not limited to adenovirus vector, an adeno-associated viral (AAV) vector, a herpes viral vector, retroviral vector, lentiviral vector, baculoviral vector.

Retroviruses are RNA viruses with a life cycle different to that of lytic viruses. In this regard, a retrovirus is an infectious entity that replicates through a DNA intermediate. When a retrovirus infects a cell, its genome is converted to a DNA form by a reverse transcriptase enzyme. The DNA copy serves as a template for the production of new RNA genomes and virally encoded proteins necessary for the assembly of infectious viral particles.

There are many retroviruses, for example murine leukemia virus (MLV), human immunodeficiency virus (HIV), equine infectious anaemia virus (EIAV), mouse mammary tumour virus (MMTV), Rous sarcoma virus (RSV), Fujinami sarcoma virus (FuSV), Moloney murine leukemia virus (Mo-MLV), FBR murine osteosarcoma virus (FBR MSV), Moloney murine sarcoma virus (Mo-MSV), Abelson murine leukemia virus (A-MLV), Avian myelocytomatosis virus-29 (MC29), and Avian erythroblastosis virus (AEV) and all other retroviridiae including lentiviruses.

A detailed list of retroviruses may be found in Coffin et al (“Retroviruses” 1997 Cold Spring Harbour Laboratory Press Eds: J M Coffin, S M Hughes, H E Varmus pp 758-763) incorporated herein by reference.

Lentiviruses also belong to the retrovirus family, but they can infect both dividing and non-dividing cells (Lewis et al (1992) EMBO J. 3053-3058) incorporated herein by reference.

The vector may be capable of transferring a polynucleotide the invention to a cell, for example a host cell as defined herein. The vector should ideally be capable of sustained high-level expression in host cells, so that the α chain and/or β chain are suitably expressed in the host cell.

The vector may be a retroviral vector. The vector may be based on or derivable from the MP71 vector backbone. The vector may lack a full-length or truncated version of the Woodchuck Hepatitis Response Element (WPRE).

For efficient infection of human cells, viral particles may be packaged with amphotropic envelopes or gibbon ape leukemia virus envelopes.

### Cell

The present invention further provides a cell e.g. a host cell comprising a polynucleotide or vector according to the invention.

The host cell may be any cell which can be used to express and produce a TCR.

Suitably, the cell is a T cell, such as a conventional T cell.

Suitably, the cell is a Treg cell.

In one aspect, the cell, such as a T cell or Treg, may be isolated from blood obtained from the subject. Suitably, the cell, such as a T cell or Treg, is isolated from peripheral blood mononuclear cells (PBMCs) obtained from the subject.

Suitably, the cell is a natural T reg which expresses FOXP3.

In one aspect, the cell is a stem cell.

In another aspect, the cell is a progenitor cell.

As used herein, the term “stem cell” means an undifferentiated cell which is capable of indefinitely giving rise to more stem cells of the same type, and from which other, specialised cells may arise by differentiation. Stem cells are multipotent. Stem cells may be for example, embryonic stem cells or adult stem cells.

As used herein, the term “progenitor cell” means a cell which is able to differentiate to form one or more types of cells but has limited self-renewal in vitro.

Suitably, the cell is capable of being differentiated into a T cell, such as a Treg.

Suitably, the cell has the ability to differentiate into a T cell, which expresses FOXP3 such as a Treg.

Suitably, the cell is a human cell. Suitable the cell is a human Treg.

Suitably, the cell may be an embryonic stem cell (ESC). Suitably, the cell is a haematopoietic stem cell or haematopoietic progenitor cell. Suitably, the cell is an induced pluripotent stem cell (iPSC). Suitably, the cell may be obtained from umbilical cord blood. Suitably, the cell may be obtained from adult peripheral blood.

In some aspects, hematopoietic stem and progenitor cell (HSPCs) may be obtained from umbilical cord blood. Cord blood can be harvested according to techniques known in the art (e.g., U.S. Pat. Nos. 7,147,626 and 7,131,958 which are incorporated herein by reference).

In one aspect, HSPCs may be obtained from pluripotent stem cell sources, e.g., induced pluripotent stem cells (iPSCs) and embryonic stem cells (ESCs).

As used herein, the term “hematopoietic stem and progenitor cell” or “HSPC” refers to a cell which expresses the antigenic marker CD34 (CD34+) and populations of such cells. In particular embodiments, the term “HSPC” refers to a cell identified by the presence of the antigenic marker CD34 (CD34+) and the absence of lineage (lin) markers. The population of cells comprising CD34+ and/or Lin(−) cells includes haematopoietic stem cells and hematopoietic progenitor cells.

HSPCs can be obtained or isolated from bone marrow of adults, which includes femurs, hip, ribs, sternum, and other bones. Bone marrow aspirates containing HSPCs can be obtained or isolated directly from the hip using a needle and syringe. Other sources of HSPCs include umbilical cord blood, placental blood, mobilized peripheral blood, Wharton's jelly, placenta, fetal blood, fetal liver, or fetal spleen. In particular embodiments, harvesting a sufficient quantity of HSPCs for use in therapeutic applications may require mobilizing the stem and progenitor cells in the subject.

As used herein, the term “induced pluripotent stem cell” or “iPSC” refers to a non-pluripotent cell that has been reprogrammed to a pluripotent state. Once the cells of a subject have been reprogrammed to a pluripotent state, the cells can then be programmed to a desired cell type, such as a hematopoietic stem or progenitor cell (HSC and HPC respectively).

As used herein, the term “reprogramming” refers to a method of increasing the potency of a cell to a less differentiated state.

As used herein, the term “programming” refers to a method of decreasing the potency of a cell or differentiating the cell to a more differentiated state.

Suitably the cell is matched or is autologous to the subject. The cell may be generated ex vivo either from a patient's own peripheral blood (1st party), or in the setting of a haematopoietic stem cell transplant from donor peripheral blood (2nd party), or peripheral blood from an unconnected donor (3rd party).

Suitably the cell is autologous to the subject. Suitably, the subject is a human.

In some aspects, the cell may be derived from ex-vivo differentiation of inducible progenitor cells or embryonic progenitor cells to the immune cell. In these instances, cells are generated by introducing DNA or RNA coding for the TCR of the present invention by one of many means including transduction with a viral vector, transfection with DNA or RNA.

Suitably, the cells are generated by introducing in addition to the TCR of the invention, DNA or RNA coding for FOXP3 by one of many means including transduction with a viral vector, or transfection with DNA or RNA.

As used herein, the term “conventional T cell” or Tconv means a T lymphocyte cell which expresses an αβ T cell receptor (TCR) as well as a co-receptor which may be cluster of differentiation 4) CD4 or cluster of differentiation 8 (CD8). Conventional T cells are present in the peripheral blood, lymph nodes, and tissues. FOXP3 is expressed by thymus derived Tregs and can be expressed by recently activated conventional T cells.

As used herein, the term “regulatory T cell” or Treg, means a T cell which expresses the markers CD4, CD25 and FOXP3 (CD4⁺CD25⁺FOXP3⁺). Tregs may also be identified using the cell surface markers CD4 and CD25 in the absence of or in combination with low-level expression of the surface protein CD127 (CD4⁺CD²⁵⁺CD127⁻). Tregs may also express on the cell surface, high levels of CTLA-4 (cytotoxic T-lymphocyte associated molecule-4) or GITR (glucocorticoid-induced TNF receptor). Unlike conventional T cells, regulatory T cells do not produce IL-2 and are therefore anergic at baseline. Treg cells include thymus-derived, natural Treg (nTreg) cells and peripherally generated, induced Treg (iTreg) cells.

In one aspect, a Treg is CD4⁺CD25⁺FOXP3⁺. T cell.

In one aspect, a Treg is a CD4⁺CD25⁺CD127⁻ T cell.

In one aspect, a Treg is a CD4⁺CD25⁺FOXP3⁺CD127⁻ T cell.

As used herein, the term “natural T reg” means a thymus-derived Treg. Natural T regs are CD4⁺CD25⁺FOXP3⁺ Helios⁺ Neuropilin 1⁺. Compared with iTregs, nTregs have higher expression of PD-1 (programmed cell death-1, pdcd1), neuropilin 1 (Nrp1), Helios (Ikzf2), and CD73. nTregs may be distinguished from iTregs on the basis of the expression of Helios protein or Neuropilin 1 (Nrp1) individually.

As used herein, the term “induced regulatory T cell” (iTreg) means a CD4⁺ CD25⁺ FOXP3⁺ Helios⁻ Neuropilin 1⁻ T cell which develops from mature CD4+ conventional T cells outside of the thymus. For example, iTregs can be induced in vitro from CD4+ CD25−FOXP3− cells in the presence of IL-2 and TGF-β.

### Compositions

The present invention also provides a composition comprising an engineered Treg, a vector or a cell according to the invention. Suitably the present invention provides a composition comprising an engineered Treg according to the invention. Suitably the present invention provides a composition comprising a vector according to the invention. Suitably the present invention provides a composition comprising a cell according to the invention.

In some embodiments, the composition is a pharmaceutical composition. Such pharmaceutical composition may comprise a pharmaceutically acceptable carrier, diluent, excipient or adjuvant. The choice of pharmaceutical carrier, excipient or diluent can be selected with regard to the intended route of administration and standard pharmaceutical practice. The pharmaceutical compositions may comprise as (or in addition to) the carrier, excipient or diluent, any suitable binder(s), lubricant(s), suspending agent(s), coating agent(s), solubilising agent(s) and other carrier agents.

The pharmaceutical compositions typically should be sterile and stable under the conditions of manufacture and storage. Formulations for parenteral administration include, but are not limited to, suspensions, solutions, emulsions in oily or aqueous vehicles, pastes, and implantable sustained-release or biodegradable formulations as discussed herein. Sterile injectable formulations may be prepared using a non-toxic parenterally acceptable diluent or solvent. A pharmaceutical composition for use in accordance with the present invention may include pharmaceutically acceptable dispersing agents, wetting agents, suspending agents, isotonic agents, coatings, antibacterial and antifungal agents, carriers, excipients, salts, or stabilizers which are non-toxic to the subjects at the dosages and concentrations employed. Preferably, such a composition can further comprise a pharmaceutically acceptable carrier or excipient for use in the treatment of disease that that is compatible with a given method and/or site of administration, for instance for parenteral (e.g. sub-cutaneous, intradermal, or intravenous injection) or intrathecal administration.

Wherein the pharmaceutical composition comprises a cell according to the invention, the composition may be produced using current good manufacturing practices (cGMP).

Suitably the pharmaceutical composition comprising a cell may comprise an organic solvent, such as but not limited to, methyl acetate, dimethyl sulfoxide (DMSO), N,N-dimethylformamide (DMF), dimethoxyethane (DME), and dimethylacetamide, including mixtures or combinations thereof.

Suitably the pharmaceutical composition comprising a cell is endotoxin free.

### Method of Treatment

The present invention provides a method for treating and/or preventing a disease which comprises the step of administering an engineered Treg of the present invention to a subject.

The present invention provides a method for treating and/or preventing a disease which comprises the step of administering a pharmaceutical composition of the present invention to a subject.

The present invention also provides an engineered Treg of the present invention for use in treating and/or preventing a disease.

The present invention also provides a pharmaceutical composition of the present invention for use in treating and/or preventing a disease.

The invention also relates to the use of an engineered Treg, a vector or cell according to the present invention in the manufacture of a medicament for treating and/or preventing a disease.

Preferably, the present methods of treatment relate to the administration of a pharmaceutical composition of the present invention to a subject.

The term “treat/treatment/treating” refers to administering an engineered Treg, cell, vector, or pharmaceutical composition as described herein to a subject having an existing disease or condition in order to lessen, reduce or improve at least one symptom associated with the disease and/or to slow down, reduce or block the progression of the disease.

Reference to “prevention”/“preventing” (or prophylaxis) as used herein refers to delaying or preventing the onset of the symptoms of the disease. Prevention may be absolute (such that no disease occurs) or may be effective only in some individuals or for a limited amount of time.

In a preferred embodiment of the present invention, the subject of any of the methods described herein is a mammal, preferably a cat, dog, horse, donkey, sheep, pig, goat, cow, mouse, rat, rabbit or guinea pig. Preferably the subject is a human.

The administration of a pharmaceutical composition of the invention can be accomplished using any of a variety of routes that make the active ingredient bioavailable. For example, a Treg, cell, vector, or pharmaceutical composition can be administered intravenously, intrathecally, by oral and parenteral routes, intranasally, intraperitoneally, subcutaneously, transcutaneously or intramuscularly.

In one aspect, the engineered Treg according to the invention or the pharmaceutical composition according to the invention is administered intravenously.

In another aspect, the engineered Treg according to the invention or the pharmaceutical composition according to the invention is administered intrathecally.

Typically, a physician will determine the actual dosage that is most suitable for an individual subject and it will vary with the age, weight and response of the particular patient. The dosage is such that it is sufficient to reduce and/or prevent disease symptoms.

Those skilled in the art will appreciate, for example, that route of delivery (e.g., oral vs intravenous vs subcutaneous, etc) may impact dose amount and/or required dose amount may impact route of delivery. For example, where particularly high concentrations of an agent within a particular site or location are of interest, focused delivery may be desired and/or useful.

Other factors to be considered when optimizing routes and/or dosing schedule for a given therapeutic regimen may include, for example, the disease being treated (e.g., type or stage, etc.), the clinical condition of a subject (e.g., age, overall health, etc.), the presence or absence of combination therapy, and other factors known to medical practitioners.

The dosage is such that it is sufficient to stabilise or improve symptoms of the disease.

The present invention also provides a method for treating and/or preventing a disease, which comprises the step of administering a pharmaceutical composition comprising a cell e.g. a T cell according to the invention to a subject.

Suitably, the present invention also provides a method for treating and/or preventing a disease, which comprises the step of administering an engineered Treg according to the invention to a subject.

The method may comprise the following steps:
(i) isolation of a cell-containing sample from a subject;
(ii) introducing a nucleic acid sequence encoding a TCR and optionally, a nucleic acid encoding a FOXP3 protein to the cells; and
(iii) administering the cells from (ii) to the subject.

Suitably the cells from (ii) may be expanded in vitro before administration to the subject.

Disease

The disease to be treated and/or prevented by the methods and uses of the present invention may be any disease which induces a T cell mediated immune response.

The disease may be, for example, a cancer, infectious disease or autoimmune disease.

Suitably the disease to be treated and/or prevented by the methods and uses of the present invention may be an autoimmune disease.

Without wishing to be bound by theory, the disease to be treated and/or prevented by the methods and uses of the present invention may be any disease wherein MBP is an antigen e.g. where MBP is a self-antigen.

Suitably the disease may be an autoimmune and inflammatory central nervous system disease (e.g. chronic neurodegenerative conditions).

Suitably the disease may be a chronic neurodegenerative condition such as multiple sclerosis (MS), Alzheimer's disease, Parkinson's disease, neurotropic viral infections, stroke, paraneoplastic disorders and traumatic brain injury.

In one aspect, the disease is multiple sclerosis.

Suitably, the disease is chronic progressive multiple sclerosis.

Suitably, the disease is relapsing/remitting multiple sclerosis.

In one aspect, the disease may have central nervous system (CNS) involvement of systemic autoimmune and inflammatory disease such as Behçet disease, sarcoidosis, systemic lupus erythematosus, juvenile idiopathic arthritis, scleroderma, and Sjögren syndrome.

Suitably, the disease is present in an HLA-DRB1*0401 positive subject.

Suitably, the disease is multiple sclerosis and the subject is HLA-DRB1*0401 positive.

Suitably, the disease is chronic progressive multiple sclerosis and the subject is HLA-DRB1*0401 positive.

Suitably, the disease is relapsing/remitting multiple sclerosis and the subject is HLA-DRB1*0401 positive.

### Multiple Sclerosis

Multiple Sclerosis (MS) is the most common neurological disorder among young adults in Europe and in the USA. MS is characterised as a demyelinating disease and is a chronic degenerative disease of the central nervous system in which gradual destruction of myelin occurs in patches throughout the brain and/or spinal cord, interfering with neural connectivity and causing muscular weakness, loss of coordination and speech and visual disturbances.

Several types or patterns of progression of MS have been identified including, clinically isolated syndrome (CIS), relapsing-remitting MS (RRMS), primary progressive MS (PPMS) and secondary progressive MS (SPMS). For some patients, the increase or progression of disability is very gradual, and for others it can occur more quickly. In general, however, recovery from attacks become less and less complete, and symptoms tend to increase and disability grows.

Although several disease-modifying treatments (DMTs) have been approved to reduce the frequency of clinical relapses, most patients continue to clinically deteriorate under current therapy schedules. Autologous haematopoietic stem cell transplantation can have lasting beneficial effects for patients, but the procedure requires aggressive myelo-ablative conditioning which is associated with substantial toxicity. Neither DMTs nor stem cell transplantation can mediate antigen-specific suppression of the immunopathology of MS. Without wishing to be bound by theory, in the future, administration of one dose of engineered Treg of the present invention may provide lasting suppression of MS immunopathology in the absence of systemic side effects. This will have a significant impact on the progression of the disease in people with MS.

Suitably, the Treg, vector or pharmaceutical composition of the present invention may reduce or ameliorate one or more of the symptoms of MS, which include reduced or loss of vision, stumbling and uneven gait, slurred speech, urinary frequency and incontinence, mood changes and depression, muscle spasms and paralysis.

### Method

The invention also provides a method for producing an engineered Treg which method comprises introducing into a cell in vitro or ex vivo, a polynucleotide encoding a TCR as defined herein. Suitably, the method further comprises incubating the cell under conditions permitting expression of the TCR molecule of the present invention. Optionally, the method may further comprise a step of purifying the engineered Treg cells.

Suitably, the cell is a T cell.

Suitably, the cell is a Treg cell.

Suitably, the cell is a natural Treg which expresses FOXP3.

In one aspect, the cell is a stem cell. Suitably, in the method according to the invention, a nucleic acid encoding TCR as defined herein has been introduced into the stem cell and the stem cell is then differentiated into a T cell such as a Treg which expresses FOXP3.

Suitably, the stem cell has the ability to differentiate into a T cell such as a Treg which expresses FOXP3. Suitably, the cell may be an embryonic stem cell (ESC). Suitably, the cell may be obtained from umbilical cord blood. Suitably, the cell may be obtained from adult peripheral blood. Suitably, the cell is a haematopoietic stem and progenitor cell (HSPC). Suitably, the cell is an induced pluripotent stem cell (iPSC).

In another aspect, the cell is a progenitor cell. Suitably the progenitor cell has the ability to differentiate into a T cell such as a Treg which expresses FOXP3.

In another aspect, the invention provides a method for producing an engineered Treg, which method comprises introducing into a cell in vitro or ex vivo a polynucleotide encoding a TCR as defined herein and a polynucleotide encoding a FOXP3 protein. Suitably the polynucleotide encoding a TCR as defined herein and the polynucleotide encoding a FOXP3 protein are provided as separate polynucleotides. Suitably the separate polypeptides are introduced separately, sequentially or simultaneously into the cell. Wherein the polypeptides are introduced separately or sequentially, suitably the polynucleotide encoding the TCR is introduced first. Wherein the polypeptides are introduced separately or sequentially, suitably the polynucleotide encoding FOXP3 is introduced first. Suitably the polynucleotide encoding a TCR as defined herein and the polynucleotide encoding a FOXP3 protein are provided on the same polynucleotide.

Suitably, the method further comprises incubating the cell under conditions causing expression of FOXP3 and the TCR molecule of the present invention. Optionally, the method may further comprise a step of purifying the engineered Treg cells.

In one aspect, the invention provides a method for producing an engineered Treg, which method comprises introducing into a cell in vitro or ex vivo a polynucleotide encoding a TCR as defined herein and a polynucleotide encoding a FOXP3 protein and differentiating the cell into a T cell, such as a Treg which expresses FOXP3. Suitably, the method further comprises incubating the cell under conditions causing expression of FOXP3 and the TCR molecule of the present invention. Optionally, the method may further comprise a step of purifying the engineered Treg cells.

Suitably, in one aspect the cell is differentiated into a T cell before FOXP3 is introduced into the cell.

Purification of the engineered Treg may be achieved by any method known in the art. Suitably, the engineered Treg may be purified using fluorescence-activated cell sorting (FACS) or immunomagnetic isolation (i.e. using antibodies attached to magnetic nanoparticles or beads) using positive and/or negative selection of cell populations.

Suitably, purification of the engineered T cell may be performed using the expression of the TCR as defined herein.

This disclosure is not limited by the exemplary methods and materials disclosed herein, and any methods and materials similar or equivalent to those described herein can be used in the practice or testing of embodiments of this disclosure. Numeric ranges are inclusive of the numbers defining the range. Unless otherwise indicated, any nucleic acid sequences are written left to right in 5′ to 3′ orientation; amino acid sequences are written left to right in amino to carboxy orientation, respectively.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure.

It must be noted that as used herein and in the appended claims, the singular forms “a”, “an”, and “the” include plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The terms “comprising”, “comprises” and “comprised of” also include the term “consisting of”.

It is noted that embodiments of the invention as described herein may be combined.

The publications discussed herein are provided solely for their disclosure prior to the filing date of the present application. Nothing herein is to be construed as an admission that such publications constitute prior art to the claims appended hereto.

The invention will now be further described by way of Examples, which are meant to serve to assist one of ordinary skill in the art in carrying out the invention and are not intended in any way to limit the scope of the invention.

### EXAMPLES

### Example 1

### Production of Retroviral Vectors Encoding TCR and TCR+FOXP3 MS2-3C8 TCR

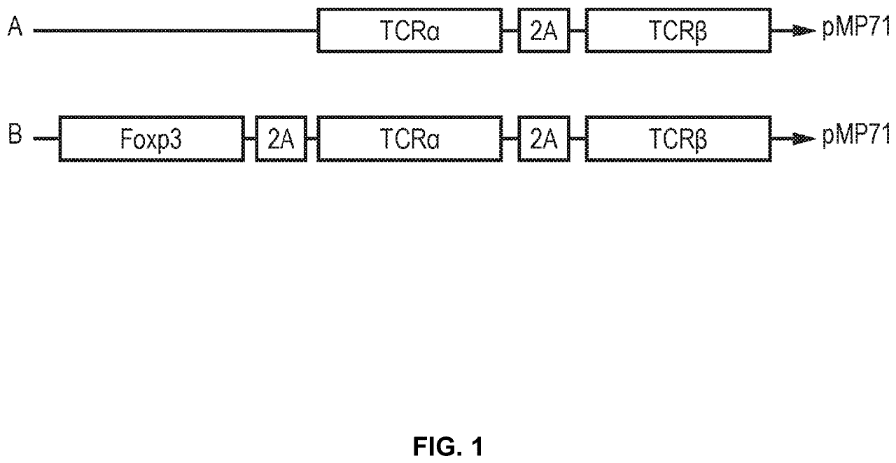

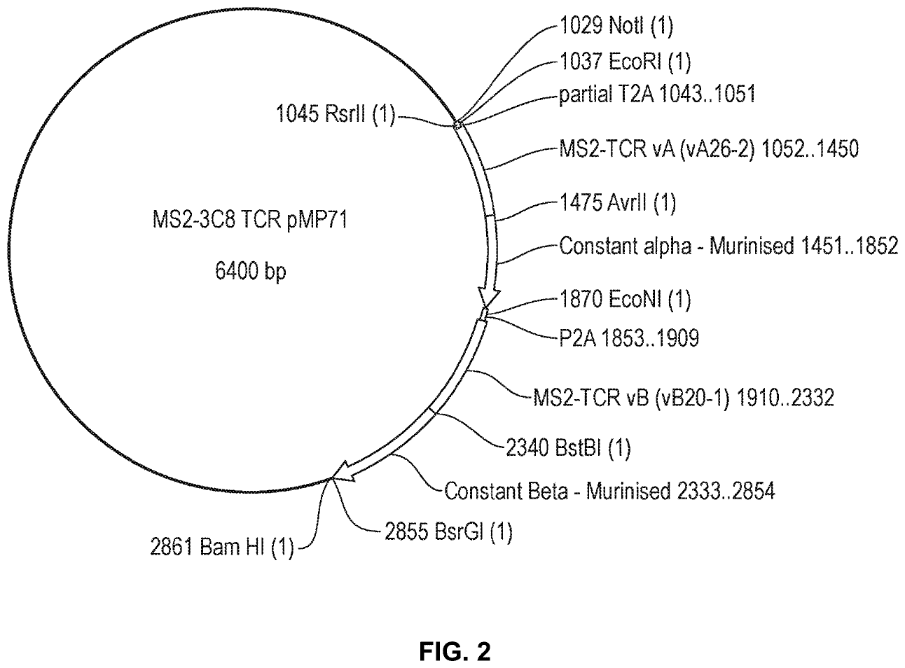

The MS2-3C8 TCR recognises MBP 111-129 (SEQ ID NO: 3) presented by HLA-DRB1*0401 as described by Muraro et al., JCI 1997; 100, 2, 339-349, incorporated herein by reference. A codon optimised MS-2 TCR expression cassette coding for the MS2-3C8 TCR was constructed (FIG. 1 and FIG. 2) by cloning the codon optimised MS2-TCR vα, murinised constant alpha domain, codon optimised MS2-TCR vβ and murinised constant beta domain into a retroviral expression cassette, pMP71. An extra disulphide bond was added between the constant-alpha and constant-beta domains as described in Blood. 2007 Mar. 15; 109 (6): 2331-2338. and Cancer Res. 2007 Apr. 15; 67 (8): 3898-3903, incorporated herein by reference. The constant-alpha and MS2-TCR vβ domains were separated by a P2A sequence. &ps MS2-3C8 FOXP3

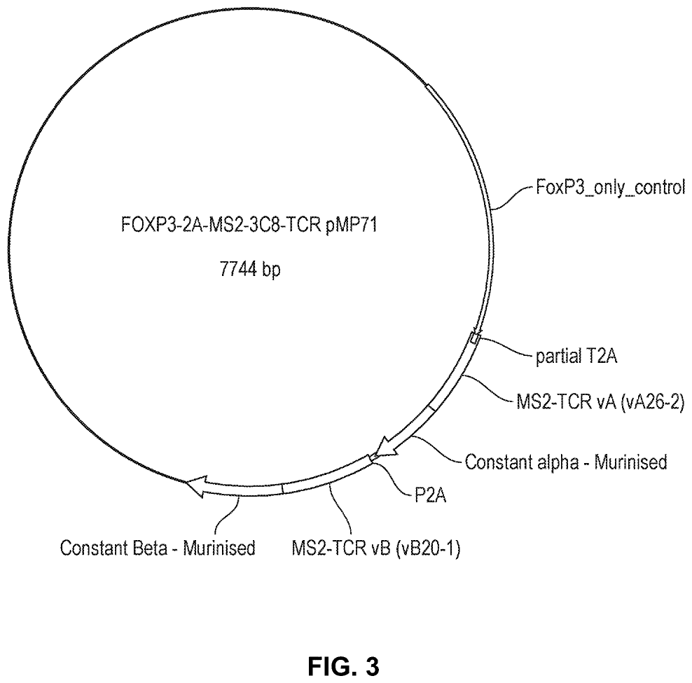

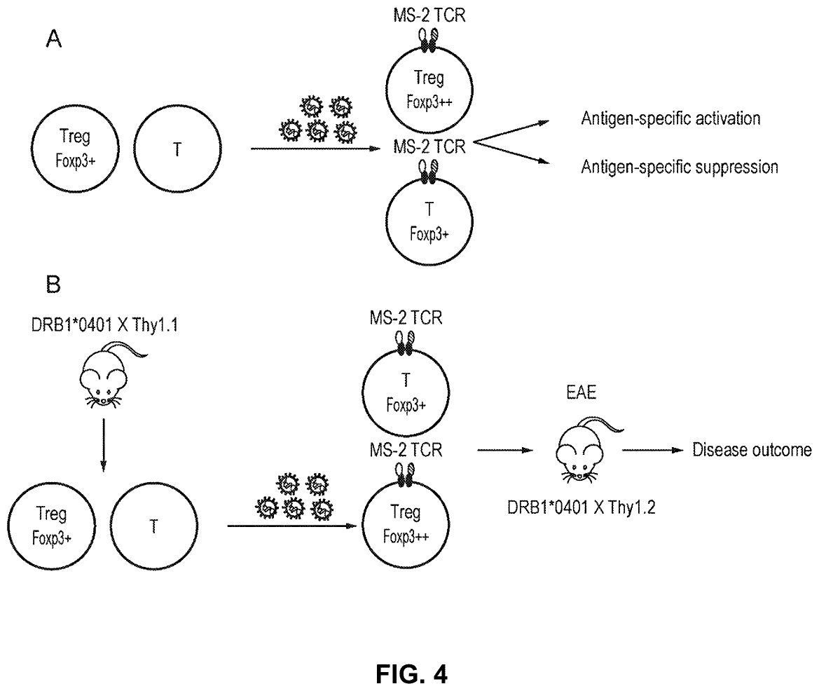

An expression cassette encoding FOXP3 and MS-2 TCR was constructed by modifying the MS-2 TCR described above (FIG. 1 and FIG. 3). A FOXP3 gene with the STOP codon removed and a T2A sequence were inserted into the pMP71 vector using the RsrII and EcoR1 sites upstream of the MS-2 TCR.

### Example 2

### Transduction of T Cells with TCR and TCR+FOXP3

Phoenix-Ampho cells were seeded at 2×10⁶ in 8 mL of Iscove's Modified Dulbecco's Media (IMDM) in standard tissue culture conditions for 24 hours. On day 1 cells were transfected using FuGENER transfection reagent and optimum media mixed with 2.5 μg of relevant plasmid DNA and 1.5 μg of pCL-Amp, encoding the ecotropic retroviral co-receptor, for 20 minutes at room temperature. The transfection mixture was added to adherent Phoenix Amphotropic (Phoenix-AMPHO) cells and incubated for a further 24 hours. On day 2 media was changed for 5 mL of TexMACS® (Miltenyi) tissue culture media and cells were incubated for a further 24 hours.

CD4+ T cells were isolated using a CD4+ Positive selection kit. Cells were subsequently stained with flow cytometry antibodies CD4, CD25 and CD127 before cell sorting using the BD FACSAria® cytometer.

CD4+CD25hiCD127− Treg and CD4+CD25-CD127+ Tconv were collected in polypropylene tubes. The purity of cell sorting was determined by addition of FOXP3 PE antibody. Purity of CD4+CD25+CD127-FOXP3+ cells was routinely >70%.

On day 0 FACS sorted cells were activated for 48 hours by culturing 1:1 with anti-CD3 and anti-CD28 beads. On day 2 cells were counted and resuspended in complete Roswell Park Memorial Institute medium (RPMI-1640) (Gibco) for Tconv or TexMACS® media for Treg at 1×10⁶/mL. Non-tissue culture-treated 24-well plates were pre-prepared by coating with retronectin, then subsequently blocked with 2% bovine serum albumin in phosphate buffered saline (PBS) and washed ×2 with PBS. The cell suspension was mixed 1:1. The final concentration of IL-2 was 300 u/ml for Tconv and 1000 u/ml for Treg. Cells were incubated overnight at 37 degrees before removing supernatant and supplementing with fresh complete media and IL-2. The media was changed on alternate days.

Tconv cells were grown in RPMI-1640 supplemented with 10% heat inactivated foetal bovine serum (FBS); 100 Units/mL penicillin; 100 μg/mL streptomycin; 2 mM L-glutamine. Regulatory T cells were cultured in TexMACX® media supplemented with 100 Units/mL penicillin; 100 μg/mL streptomycin.

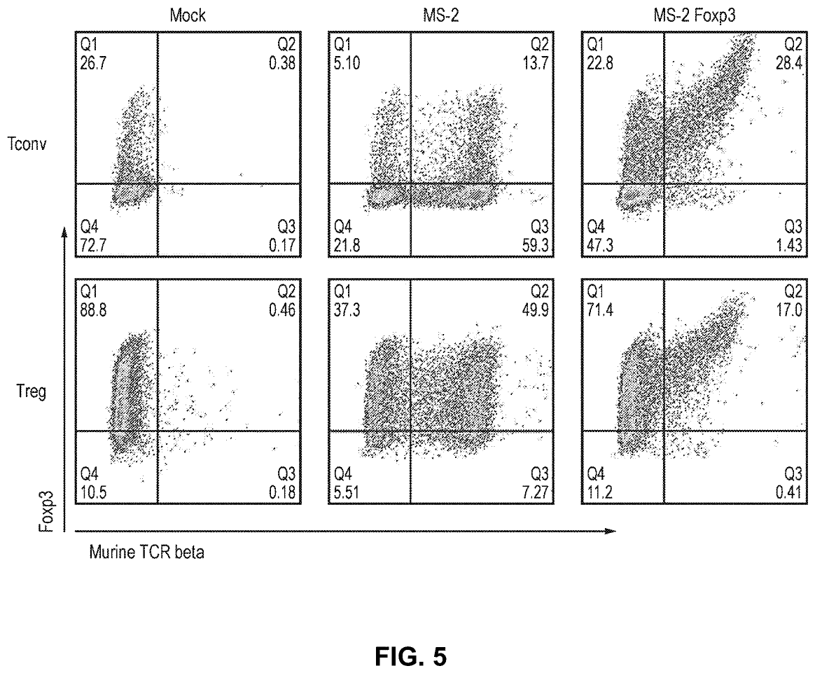

The FACS dot-plots in FIG. 5 show representative flow cytometric analysis performed at day 7 to assess the level of transduction through measurement of the expression of murine TCR constant regions and FOXP3.

### Example 3

### FOXP3 Expression During In Vitro Expansion

T cells were isolated as described above in Example 2. At day 0 the expression of FOXP3 and Treg cell surface markers CTLA-4 (also known as CD152) and CD25 was measured by flow cytometry.

T cells were transduced and cultured as described above in Example 2. At day 7 the cells were analysed by flow cytometry and the dot-plots were gated on transduced cell populations. The relative expression of FOXP3, CTLA-4 and CD25 was measured. FIG. 6 shows bar charts depicting expression of FOXP3, CTLA-4 and CD25 at day 7 of in vitro expansion. FOXP3 expression is maintained during in vitro expansion.

### Antigen-Specific Activation of MS-2 TCR Transduced T Cells

### Example 4

### Peptide Restimulation of Effector T Cells and Tregs

Chinese Hamster Ovary (CHO) cells were transduced with human HLA-DR4 and CD80 or CD86. Cells expressing CD80 or CD86 were mixed together in equal parts for subsequent experiments.

CHO cells were resuspended at 10×10⁶/mL in culture media with saturating amounts (10 μM/ml) of MBP 111-129 (SEQ ID NO: 3) (LSRFSWGAEGQRPGFGYGG). Suspensions were incubated for 2 hours at standard tissue culture conditions before being irradiated, washed and resuspended.

T cells transduced with MS2 or MS2-FOXP3 construct were washed, counted and resuspended at 0.5×10⁶ cells/ml in complete RPMI. Cells were plated 1:1 with CHO cells incubated with or without peptide for 4 hours. Cells were fixed and permeablised before staining with antibodies for IL-2 and IFNγ.

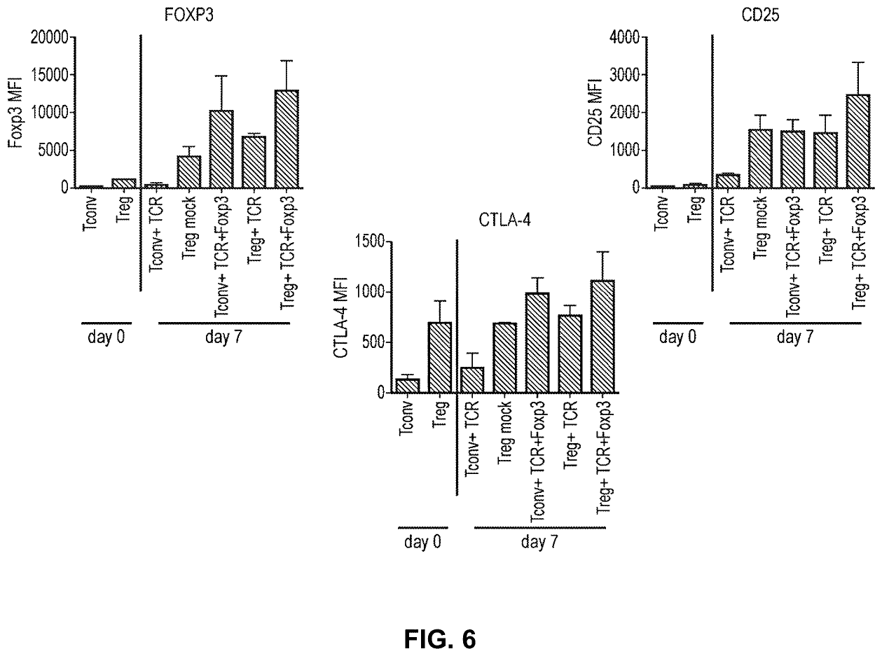

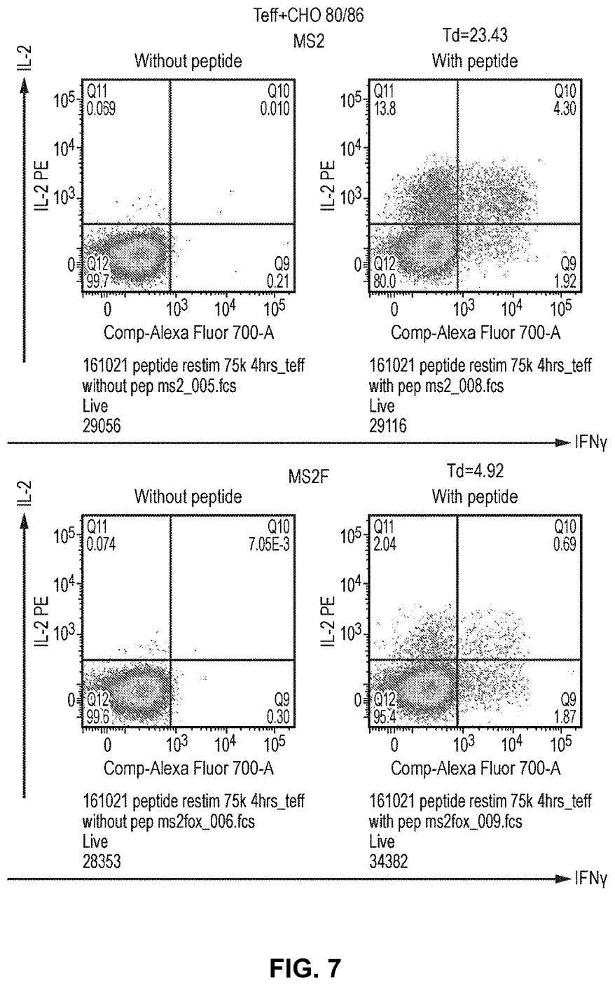

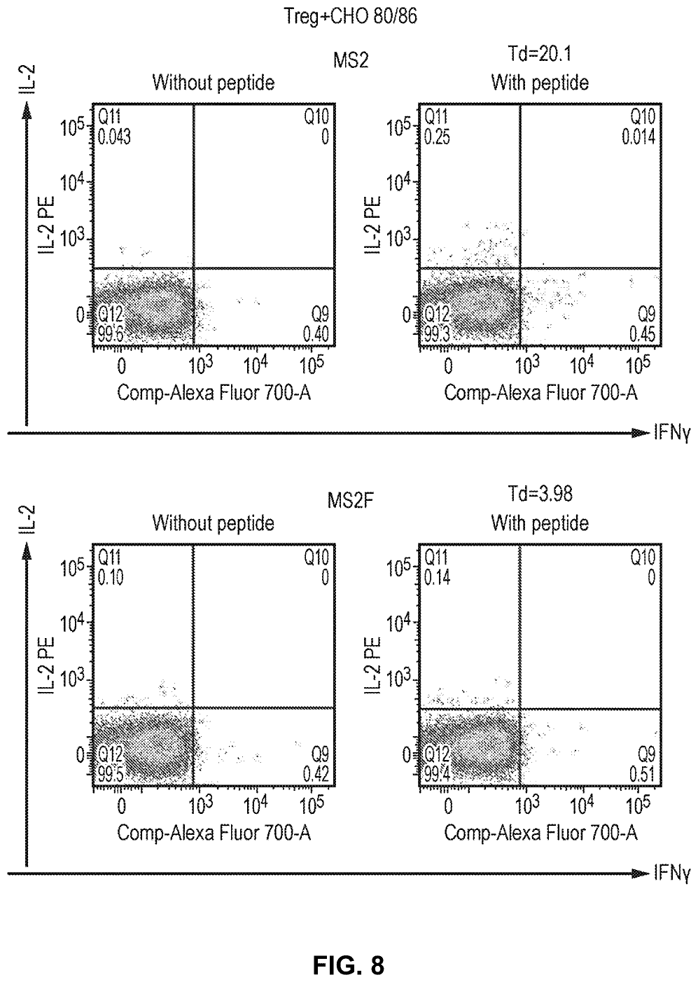

FIG. 7 demonstrates FACS dot-plots showing peptide restimulation of effector T cells. The transduction efficiency of T cells is indicated by ‘Td=’. Treg cells were cultured with CHO cells as described above. FIG. 8 demonstrates FACS dot-plots showing peptide restimulation of Treg cells. The transduction efficiency of T cells is indicated by ‘Td=’.

### Example 5

### TCR-Transduced and TCR-FOXP3 Transduced T Cells Response to Cognate Peptide

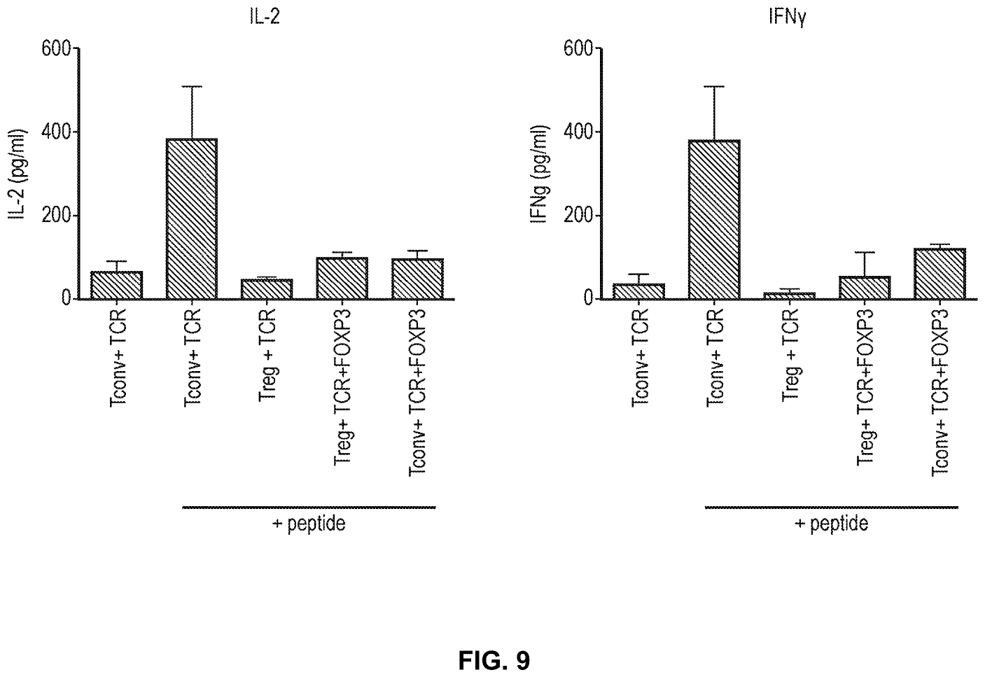

TCR-transduced T conv, TCR-transduced Tregs, TCR-FOXP3 converted Tconv and TCR-FOXP3 converted Tconv (methods described above) were cultured for 4 days with or without pepide-pulsed irradiated APC. Supernatant was collected from the culture and assayed for IL-2 and IFNγ by ELISA (n=2-4). FIG. 9 shows that TCR-transduced Treg and TCR-FOXP3 converted Tconv response to cognate peptide.

### Example 6

### TCR-Transduced and TCR-FOXP3 Transduced

CHO cells were prepared as described above. T conv cells were transduced with TCR or with TCR+FOXP3. Transduced T cells were isolated by magnetic bead sorting. Transduced cells were stained with an anti-murine constant beta antibody conjugated to APC. Cells were thoroughly washed and stained with a second anti-APC antibody. Cells were washed and passed through a magnetic column and transduced cells were captured and eluted. Routinely >95% of purified cells were APC+.

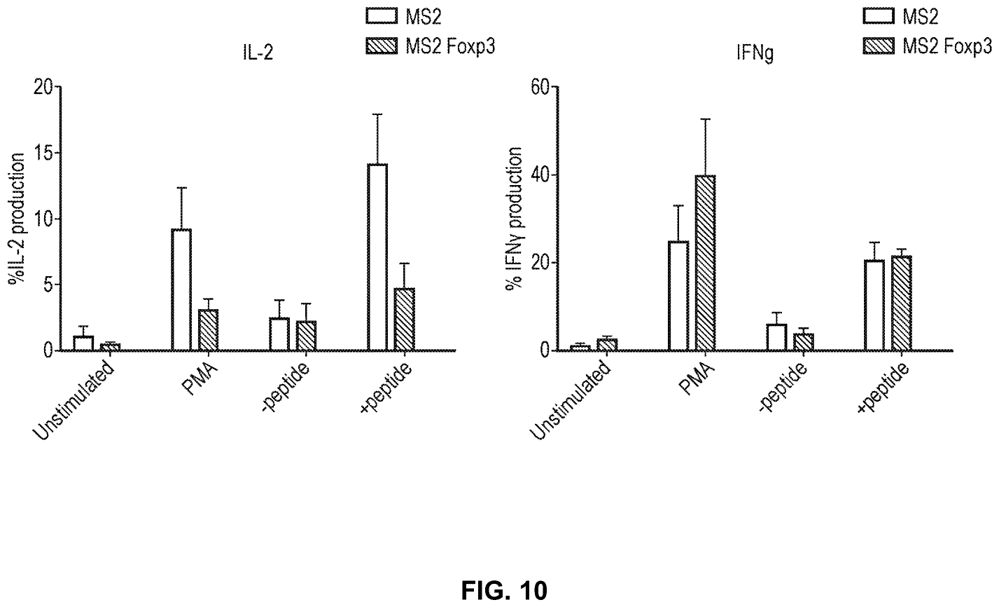

Unstimulated cells cultured without CHO cells acted as a negative control. PMA (phorbol 12-myristate 13-acetate) stimulated cells acted as a positive control. White bars show cytokine production from cells expressing the MS2 TCR and black bars show cytokine production from T cells expressing MS2 TCR and FOXP3 (n=3). FIG. 10 shows that conventional T cells transduced with TCR and TCR+FOXP3 produce less IL-2 than conventional cells transduced with TCR alone.

### Antigen-Specific Suppression of MS-2 TCR Transduced Tregs

### Example 7

### Proliferation of TCR-Transduced T Cells

CD80+CD86+DR4+ CHO cells were loaded with peptide and irradiated as described above before being resuspended at 0.1×10⁶ cells/ml. Transduced responder T cells were stained with CFSE cell trace dye in warmed PBS at 37 degrees for 3 minutes before addition of equal volumes of warm FBS and a further 3 minute incubation.

Cells were washed in 5× volume of complete media before being counting and resuspended at 1×10⁶ transduced cells/ml. The transduction efficiency of Tconv and Treg were determined by flow cytometry. Regulatory T cells are removed from culture, washed and resuspended at 1×10⁶ transduced cells/ml in complete RPMI. Cells were plated 1 Treg:0.1 CHO cells: and varying ratios of Tconv. Proliferation was determined by analysing dilution of carboxyfluorescein succinimidyl ester (CFSE)-stained T conv.

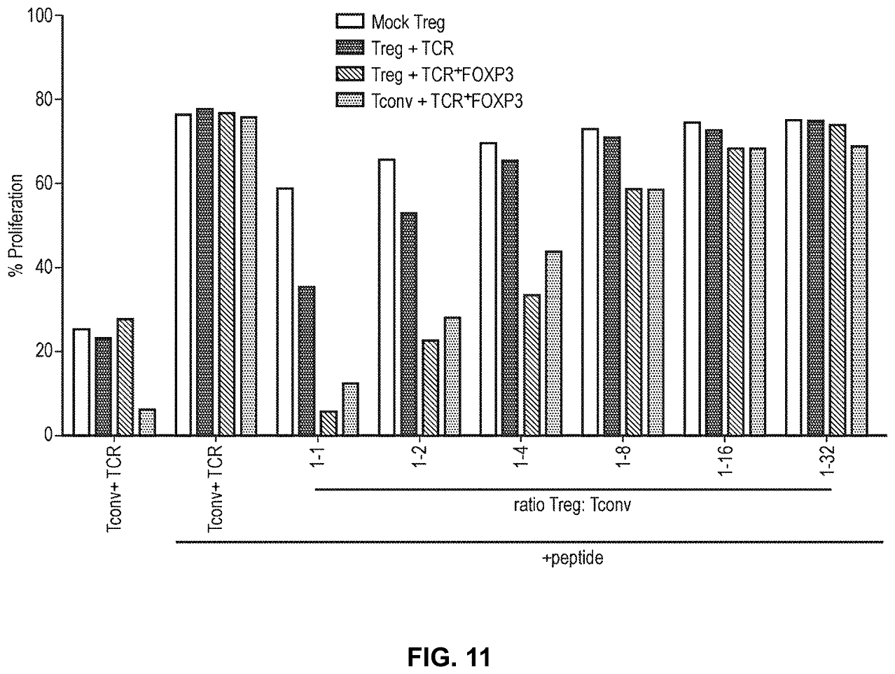

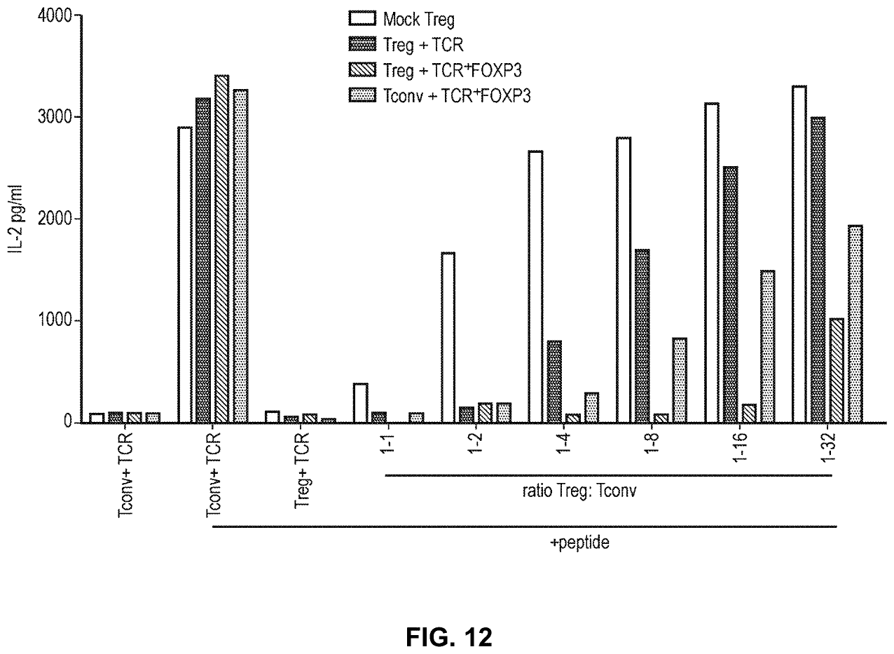

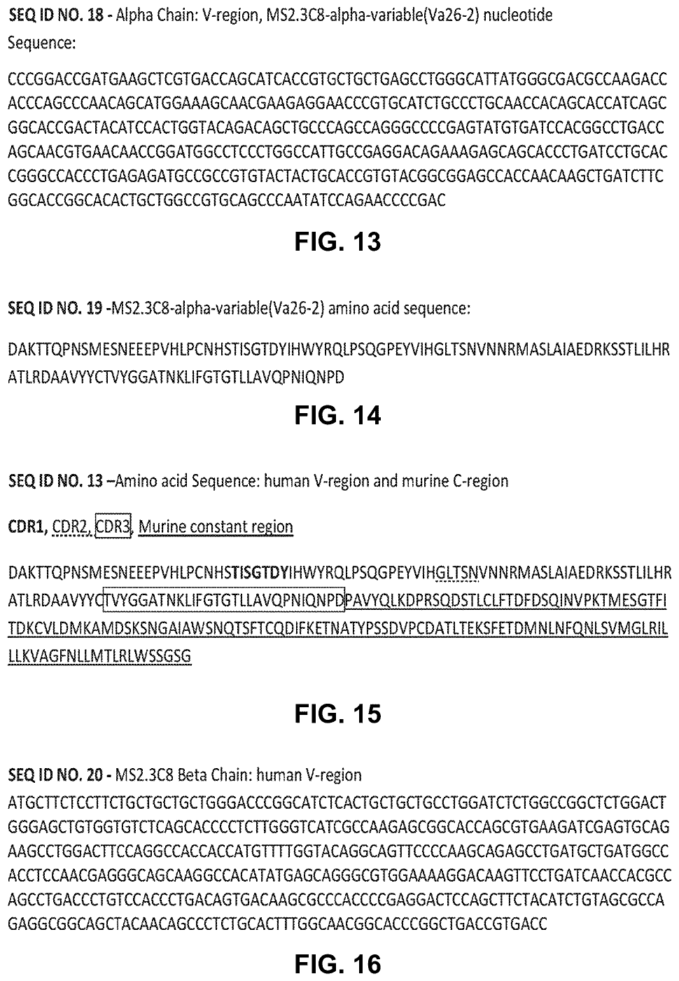

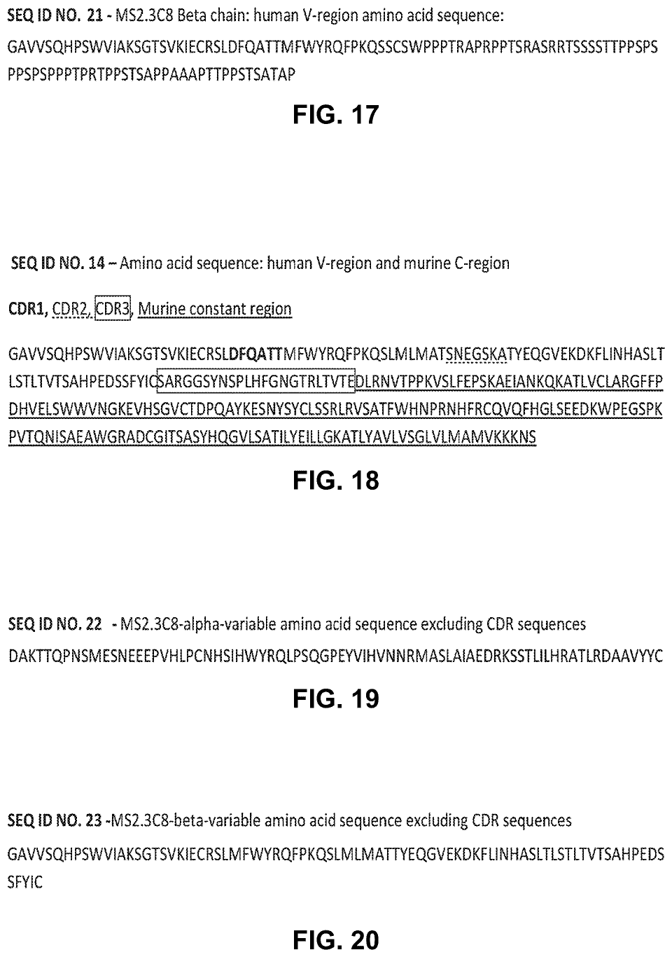

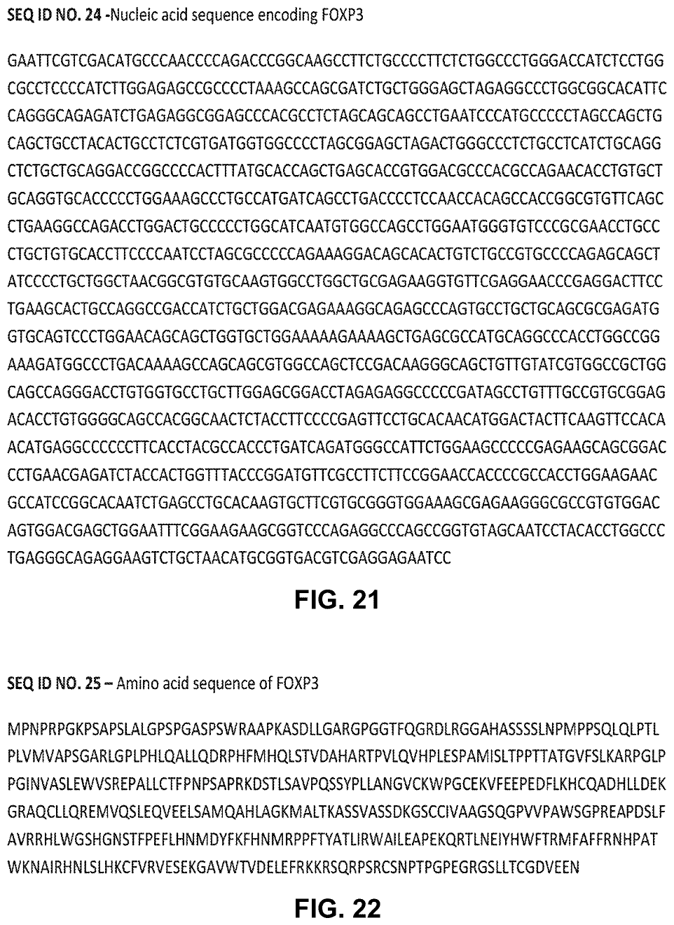

The data in FIG. 11 show that TCR-transduced Tregs suppress proliferation in an antigen-specific manner. Supernatants were collected from the culture media and were assayed for IL-2 by ELISA. The data presented in FIG. 12 show that TCR-transduced Treg suppress IL-2 production in an antigen-specific manner.

### Example 8

### Engineered Tregs in an Adoptive Transfer Model of Experimental Autoimmune Encephalomyelitis

Human conventional T cells which have been transduced with MS2-3C8 TCR are administered to immunodeficient NOD scid gamma mice (NSG) transgenic for HLADRB1*0401 by adoptive transfer.

Adoptive transfer of the human T cells may induce an experimental autoimmune encephalomyelitis (EAE) type disease in the mice. The mice are then treated with human Treg cells which have been transduced with MS2-3C8 TCR or control Tregs.

HLA-DRB1+0401-restricted MBP 111-129 (SEQ ID NO: 3)-specific humanised TCR transgenic mice have infiltrates of MS2-3C8 transgenic T cells and inflammatory legions located in the brainstem and the cranial nerve roots in addition to the spinal cord and spinal nerve roots (Quandt et al., J. Exp. Med. V. 200 (2);2004 incorporated herein by reference).

The suppression of proliferation of pathogenic T cells by TCR induced Tregs is measured in the mouse model e.g. by CFSE, IL-2 and/or IFNγ levels.

### Example 9

### Engineered Tregs in Classical Model of Experimental Autoimmune Encephalomyelitis

Mice are immunised with Mog (myelin oligodendrocyte protein) to induce EAE, a widely accepted animal model of MS.

The mice are then treated with human Treg cells which have been transduced with MS2-3C8 TCR or control Tregs. The suppression of proliferation of pathogenic T cells by TCR induced Tregs is measured in the mouse model e.g. by CFSE, IL-2 and/or IFNγ levels.

### Example 10

### TCR Transduced Regulatory T Cells can Engraft into Irradiated Hosts

CD4+CD25+ Treg were isolated from lymph nodes and splenocytes of HLA-DRB*0401 transgenic mice by bead sort. Treg were transduced with TCR. 1 day after transduction TCR or TCR+FOXP3 transduced cells were injected into HLA-DRB*0401 transgenic hosts conditioned with 4Gy irradiation (day 0). 7 weeks later flow cytometry was used to determine the engraftment of transduced Treg via staining for TCR (+7 weeks).

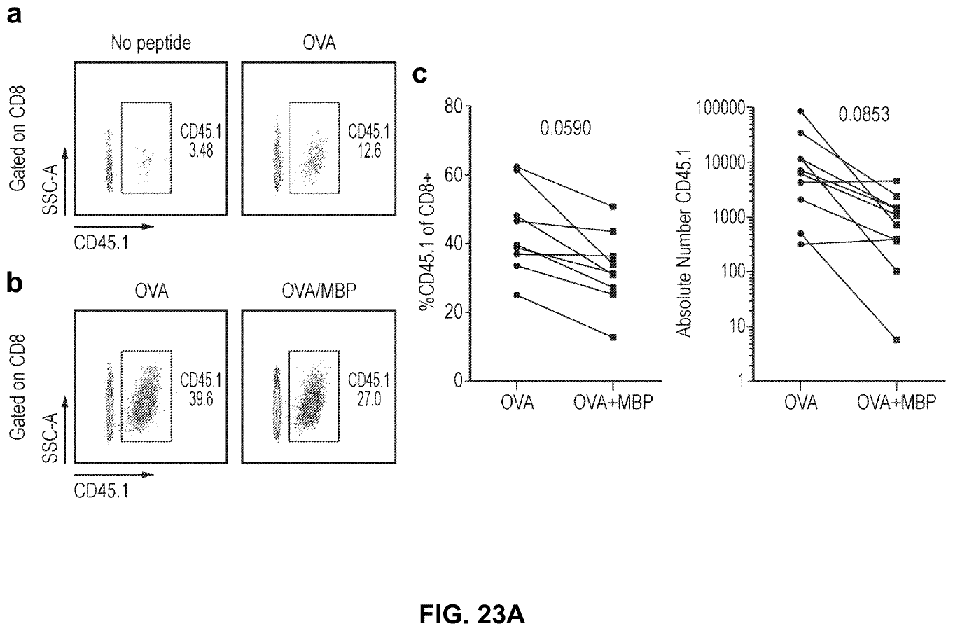

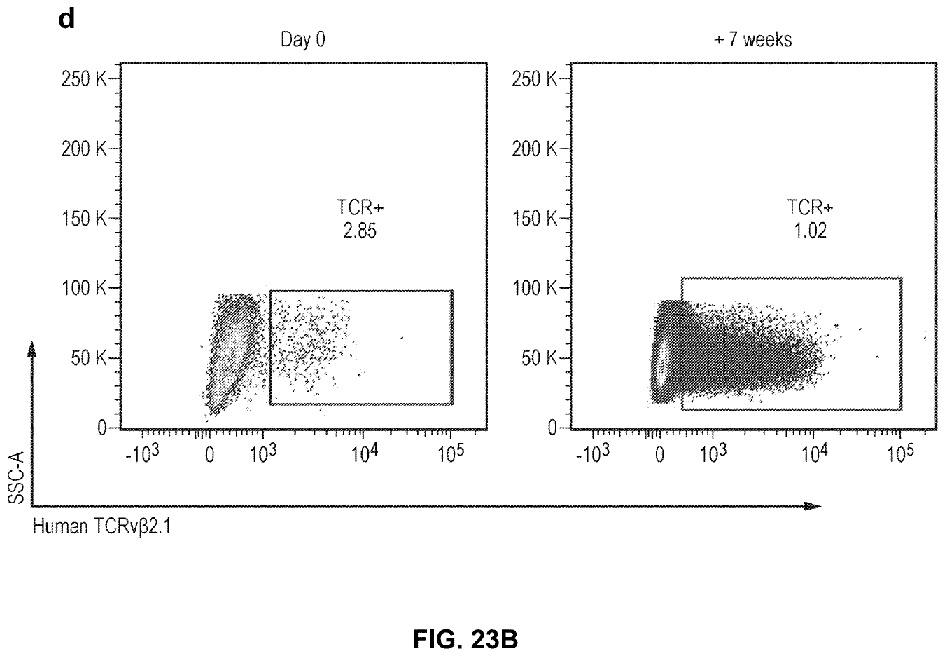

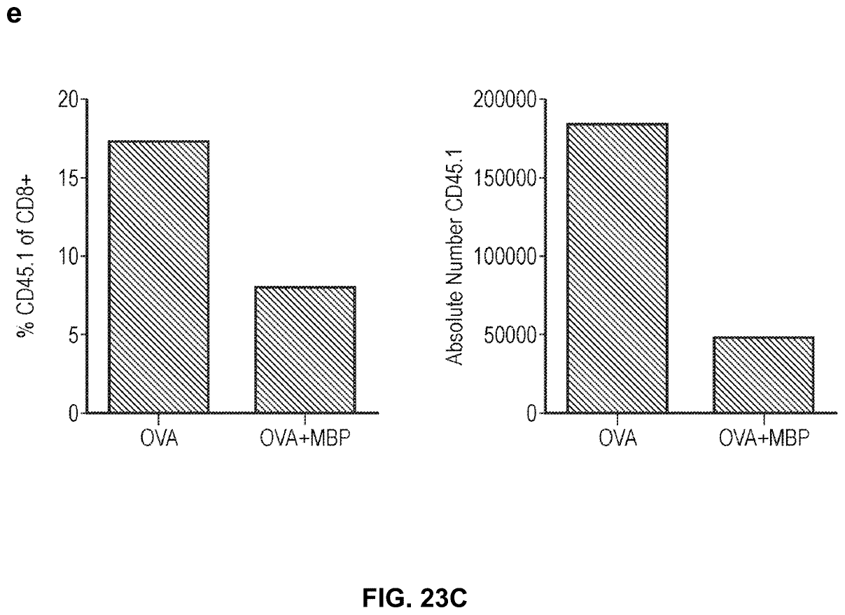

The results in FIG. 23 show persistence of Tregs and an antigen-specific suppressive effect 7 weeks post-administration.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology, cellular immunology or related fields are intended to be within the scope of the following claims.

1. An engineered regulatory T cell (Treg) comprising a T cell receptor (TCR) which is capable of specifically binding to a peptide which comprises MBP 111-129 (SEQ ID NO: 3) or variant with at least 90% identity to SEQ ID NO: 3 or a fragment thereof when the peptide is presented by a major histocompatibility complex (MHC) molecule.

2. An engineered Treg according to claim 1, wherein the peptide is capable of being presented by a HLA-DRB1*0401 molecule.

3. An engineered Treg according to any preceding claim, wherein the TCR comprises an α chain and a β chain,
wherein the α chain and the β chain each comprises three complementarity determining regions (CDRs) and the sequence of each CDR3 is as follows:
CDR3α
(SEQ ID NO: 5)
TVYGGATNKLIFGTGTLLAVQPNIQNPD
CDR3β
(SEQ ID NO: 6)
SARGGSYNSPLHFGNGTRLTVTE
or a variant of those sequences having up to three amino acid changes.

4. An engineered Treg according to claim 3, wherein the α chain of the TCR comprises three CDRs having the following amino acid sequences:
CDR1α
(SEQ ID NO: 7)
TISGTDY
CDR2α
(SEQ ID NO: 8)
GLTSN
CDR3α
(SEQ ID NO: 9)
TVYGGATNKLIFGTGTLLAVQPNIQNPD
or variants of those sequences having up to three amino acid changes;
and wherein the β chain of the TCR comprises three CDRs having the following amino acid sequences:
CDR1β
(SEQ ID NO: 10)
DFQATT
CDR2β
(SEQ ID NO: 11)
SNEGSKA
CDR3β
(SEQ ID NO: 12)
SARGGSYNSPLHFGNGTRLTVTE
or variants of those sequences having up to three amino acid changes.

5. An engineered Treg according to claim 4, wherein:
a) the variable region of the α chain of the TCR comprises an amino acid sequence having at least 80% sequence identity to SEQ ID NO: 19, wherein the sequence identity does not include the CDR sequences as defined in claim 4; and
(b) the variable region of the β chain of the TCR comprises an amino acid sequence having at least 80% sequence identity to SEQ ID NO: 21, wherein the sequence identity does not include the CDR sequences as defined in claim 4.

6. An engineered Treg according to any of claims 1 to 4, wherein:
(a) the variable region of the α chain of the TCR comprises an amino acid sequence having at least 80% sequence identity to SEQ ID NO: 19; and
(b) the variable region of the β chain of the TCR comprises an amino acid sequence having at least 80% sequence identity to SEQ ID NO: 21.

7. An engineered Treg according to any preceding claim, wherein the constant region domains of the α chain and β chain of the TCR each comprise an additional cysteine residue, enabling the formation of an extra disulphide bond between the α chain and the β chain.

8. An engineered Treg according to any preceding claim wherein:
(a) the α chain of the TCR comprises an amino acid sequence having at least 80% sequence identity to SEQ ID NO: 13; and
(b) the β chain of the TCR comprises an amino acid sequence having at least 80% sequence identity to SEQ ID NO: 14.

9. An engineered Treg according to any preceding claim, wherein the Treg is derived from a T cell isolated from a subject.

10. A pharmaceutical composition comprising an engineered Treg according to any preceding claim.

11. An engineered Treg or pharmaceutical composition according to any preceding claim for use in treating a disease.

12. The use of an engineered Treg or pharmaceutical composition according to any of claims 1 to 10 in the manufacture of a medicament.

13. A method for treating or preventing a disease in a subject in need of same which comprises the step of administering an engineered Treg or pharmaceutical composition according to any of claims 1 to 10 to the subject.

14. An engineered Treg or pharmaceutical composition for use, a use or a method according to any of claims 11 to 13 wherein the disease is multiple sclerosis.

15. An engineered Treg or pharmaceutical composition for use, a use or a method according to any of claims 11 to 14 wherein the subject is an HLADRB1*0401 positive subject.

16. A vector which comprises a nucleic acid sequence which encodes a TCR as defined in any one of claims 1 to 9 and a nucleic acid sequence which encodes FOXP3.

17. A kit of polynucleotides or vectors which comprises a first polynucleotide or vector which comprises a nucleic acid sequence which encodes a TCR as defined in any one of claims 1 to 9 and a second polynucleotide or vector which comprises a nucleic acid sequence which encodes FOXP3.

18. A method for producing an engineered Treg according to any of claims 1 to 9 which comprises the step of introducing into a cell in vitro or ex vivo a polynucleotide encoding a TCR as defined in any of claims 1 to 9.

19. A method according to claim 18 wherein the method further comprises the step of introducing into the cell in vitro or ex vivo a polynucleotide encoding a FOXP3 protein.

20. A method according to claim 18 wherein the cell is a T cell.

21. A method according to claim 20 wherein the T cell is a natural Treg which expresses FOXP3.

22. A method according to claim 20 wherein the T cell is a conventional T cell.

23. A method according to claim 19 or claim 22 wherein the step of introducing the polynucleotide encoding a TCR and the polynucleotide encoding FOXP3 are performed sequentially, separately or simultaneously.

24. A method according to claim 23 wherein the polynucleotide encoding a TCR and the polynucleotide encoding FOXP3 are introduced to the cell using the vector of claim 16.
